(19) United States
(12) Patent Application Publication
(10) Pub. No.: US 2012/0225083 A1
(43) Pub. Date: Sep. 6, 2012

(54) VIRAL POLYPEPTIDES AND METHODS

(75) Inventors: Damian Purcell, North Balwyn VIC (AU); Robert Center, Rosanna VIC (AU); Sharmila Mohana Rama Reddy, Carlton VIC (AU); Anthony Dominic Kelleher, Bangor NSW (AU); David Albert Cooper, Bellevue Hill NSW (AU); Paul Rene Gorry, Vermont VIC (AU); Jasminka Sterjovski, Brunswick VIC (AU)

(73) Assignees: THE UNIVERSITY OF MELBOURNE, PARKVILLE, VICTORIA (AU); THE MACFARLANE BURNET INSTITUTE FOR MEDICAL RESEARCH AND PUBLIC HEALTH, MELBOURNE VICTORIA (AU); NEWSOUTH INNOVATIONS PTY LIMITED, SYDNEY, NEW SOUTH WALES (AU)

(21) Appl. No.: 13/119,419

(22) PCT Filed: Sep. 16, 2009
(86) PCT No.: PCT/AU2009/001218

§ 371 Date: Jul. 6, 2011

(30) Foreign Application Priority Data
Sep. 16, 2008 (AU) 2008904790

(51) Int. Cl. A61K 39/21; A61P 31/18; A61K 38/16; A61K 39/42; C07K 14/16; C07K 16/10

(52) U.S. Cl. 424/160.1; 530/350; 530/389.4; 514/3.8; 424/208.1

(57) ABSTRACT

The invention is directed to polypeptides and polypeptide fragments from HIV-1 envelope (Env) proteins following the characterization of Env structures from environments where HIV isolates expose conserved neutralization-sensitive Env structures. There is provided a polypeptide being all or a fragment of an HIV-1 envelope protein from a transmission strain of HIV-1, the polypeptide having neutralization sensitive epitopes that are accessible.

### FIELD OF THE INVENTION

The invention relates to HIV-1 envelope glycoprotein variants, antibodies to those variants, and the use of the glycoprotein variants and antibodies in vaccines and methods of reducing the risk of infection with HIV-1 and treating HIV-1 infection.

### BACKGROUND OF THE INVENTION

Entry of enveloped viruses into cells requires transformation of the protective envelope into a fusion-competent state. In the case of the human immunodeficiency virus (HIV-1), the gp120 and gp41 envelope glycoprotein (Env) complexes mediate viral entry into cells. Infection is initiated by the selective interaction between the viral envelope glycoprotein, gp120, and receptors on the target cell, CD4. The elaborately structured trimer of gp120 subunits bind to CD4 which induces conformational changes that lead to exposure of a binding site for a cellular coreceptor, typically CCR5 or CXCR4, but also other chemokine coreceptors. Coreceptor binding transmits further conformational changes to gp41 that lead to fusion between the viral and cellular membranes and entry of the HIV-1 core into the cell.

Both humoral and cellular immune approaches have been pursued to develop vaccine candidates for both prophylactic and therapeutic use. The envelope of HIV is the only surface of the virus accessible for humoral immune responses that prevent infection and receptor binding regions of gp120 are potentially key neutralising epitopes shared between diverse HIV strains. Despite this, modifications aimed at exposing neutralising epitopes with a view to eliciting broadly neutralising antibodies (Nabs) against HIV-1 have, to date, had little or no success. Accordingly, there is a need to identify novel candidate immunogens that prominently present neutralising epitopes which can elicit humoral immune response that include broad Nabs and confer protection against HIV infection or represent a new target for the treatment of HIV infection.

### SUMMARY OF THE INVENTION

The invention is directed to polypeptides and polypeptide fragments from HIV-1 envelope (Env) proteins following the characterisation of Env structures from environments where HIV isolates expose conserved neutralisation-sensitive Env structures. Accordingly, in one aspect of the invention, there is provided a polypeptide being all or a fragment of an HIV-1 envelope protein from a transmission strain of HIV-1, the polypeptide having neutralisation sensitive epitopes that are accessible.

The inventors surprisingly discovered that the transmission strains of HIV-1 have differences in their Envs including fewer N-linked glycans and shorter variable loops. In a preferred embodiment of this aspect of the invention, the envelope protein from the transmission strain has an altered glycosylation profile compared to a glycosylation profile of an envelope protein from post-seroconversion strains of HIV-1. The altered glycosylation profile may be as a result of deletion, modification, or mutation of one or more glycosylation sites within the HIV-1 envelope polypeptide.

In an alternative embodiment the envelope protein from the transmission strain has one or more altered variable loop sequences compared to variable loop sequences of an envelope protein from post-seroconversion strains of HIV-1. Comparison may be made to variable loop sequences of an envelope protein from post-seroconversion strains of HIV-1 having the same function or having at least 95% sequence identity.

In yet another embodiment, the envelope protein from the transmission strain has both an altered glycosylation profile and one or more altered variable loop sequences compared to a glycosylation profile and variable loop sequences of an envelope protein from post-seroconversion strains of HIV-1.

The invention also provides a polypeptide comprising a fragment of an HIV-1 envelope protein from a transmission strain of HIV-1, the polypeptide having neutralisation sensitive epitopes that are accessible.

Having determined that transmission strains of HIV have an altered glycosylation profile with fewer epitopes being “masked” by glycans, and that transmission strains may also have epitopes exposed due to a different gp120 conformation as a result of altered variable loops, the inventors used these epitopes to elicit neutralising antibodies.

Accordingly, in another aspect of the invention, there is provided an HIV-1 neutralising antibody for binding neutralisation sensitive epitopes, wherein the neutralisation sensitive epitopes are accessible in a transmission strain of HIV-1. Preferably the polypeptide has either or both an altered glycosylation profile and an altered variable loop sequence.

In another aspect of the invention there is provided an oligomeric polypeptide comprising a polypeptide of the invention wherein the oligomeric polypeptide generates neutralising antibodies to transmission strains of HIV-1, the transmission strain preferably having either or both an altered glycosylation profile and one or more altered variable loop sequences of an envelope protein from post-seroconversion strains of HIV-1. In one embodiment of this aspect of the invention, the oligomeric polypeptide comprises a polypeptide being all or a fragment of an HIV-1 envelope protein from a transmission strain of HIV-1, wherein the envelope protein lacks a hydrophobic transmembrane anchored region.

The polypeptides of the invention can be utilised in methods to reduce the risk of HIV infection in seronegative individuals, or to treat HIV-1 seropositive individuals to minimise symptoms of HIV-1 infection. The methods include the step of administering to the individual an HIV-1 envelope polypeptide or polypeptide fragment wherein one or more glycosylation sites in gp120, gp41, or both are deleted or mutated.

In an alternative embodiment, the method include administering to the individual a polypeptide being all or a fragment of an HIV-1 envelope protein from a transmission strain of HIV-1, the polypeptide having neutralisation sensitive epitopes that are accessible.

The methods of the invention can include the administration of either or both the neutralising antibodies of the invention and the oligomeric polypeptides of the invention in addition to or instead of the polypeptides.

The polypeptides of the invention contain important targets for neutralising antibodies, and can themselves serve as immunogens that can stimulate the production of neutralising antibodies. Accordingly, in another aspect of the invention, there is provided a composition for use in raising an immune response comprising an HIV-1 envelope polypeptide or polypeptide fragment wherein one or more glycosylation sites in gp120, gp41, or both are deleted or mutated.

In an alternative embodiment of this aspect of the invention, the composition for use in raising an immune response includes a polypeptide being all or a fragment of an HIV-1 envelope protein from a transmission strain of HIV-1, the polypeptide having neutralisation sensitive epitopes that are accessible.

Compositions of the invention can also include either or both the neutralising antibodies of the invention and the oligomeric polypeptides of the invention in addition to or instead of the polypeptides.

### DESCRIPTION OF THE INVENTION

It will also be understood that the term “comprises” (or its grammatical variants) as used in this specification is equivalent to the term “includes” and should not be taken as excluding the presence of other elements or features.

The humoral immune response against HIV-1 is mainly characterised by neutralising antibodies (Nabs). In patients with long term HIV infection, who show no or slow clinical progress of infection, broadly cross-reacting Nabs are found and are protective. Similarly, HIV-1 specific IgA response is protective in the case of exposed seronegative individuals. However, in progressive infection only low titers of Nabs are seen and there is rapid virus escape from these Nabs leading to poor or no protection. HIV-1 also shields the conserved structures of Env through various strategies including glycosylation, hiding using variable loops (including varying the length of the loops as well as sequence), transient exposure and conformational masking leading to inefficient Nab responses.

These Nabs act by binding the HIV-1 Env trimer spike and impeding the attachment of HIV-1 gp120 surface envelope (Env) protein to target cell CD4 and/or chemokine co-receptor molecules or by disrupting the fusion mechanism of the Env transmembrane protein (gp41). Other antibodies may protect from infection by promoting cell-mediated responses to virus infected cells through the antibody-dependent cellular toxicity (ADCC) mechanism. There is vast amino-acid sequence and conformational diversity of HIV-1 Env across viral strains.

The inventors characterised Env structures from environments where HIV isolates that expose conserved neutralisation-sensitive Env structures may thrive. The inventors surprisingly discovered that transmission strains of HIV-1 have differences in their Envs including fewer N-linked glycans and shorter variable loops. By “transmission strain” it is meant the earliest, or one of the earliest, strains of HIV-1 present in the infected individual that exist before an adaptive immune response. As infection progresses, there is consistent accumulation of N-linked glycans and variable loop sequence mainly driven by humoral neutralisation pressure and escape from T-cell responses. By the time the infection is established, potential neutralising epitopes of Env tend to have been masked due to the increased degree of glycosylation and/or the different oligomeric structure of Env due to altered variable loops, and are therefore no longer exposed to or accessible to broad Nabs, or indeed, are able to generate Nabs, or indeed, are able to generate Nabs. These same amino acid changes can alter the presentation or recognition of peptides to cell-mediated immunity. Transmission strains (also referred to as “pre-seroconversion strains”) are selected from the donor's viral swarm by highly efficient receptor engagement and fusion and their Env therefore tend to better functional components such as receptor binding sites through fewer N-linked glycans and shorter variable loops when compared to post-seroconversion strains of HIV-1. Common alterations occur at one or more of N197, N358 and N386 in HIV-1 strains (based on the numbering of the HIV-1 strain HXB2 according to accession number K03455 version K03455.1, and as shown in SEQ ID NO: 36).

It will be appreciated that pre-seroconversion strains may be present in individuals who have seroconverted. Site within the body (known as “sanctuary sites”), such as the brain and testis, are not subject to the same immune exposure. Consequently, despite seroconversion, strains of the virus with fewer N-linked glycans and shorter variable loops can that prominently expose neutralisation-sensitive functional components of Env remain in the infected individual.

By “post-seroconversion strains” it is meant that the individual from whom the virus strain was isolated has developed serological evidence of HIV-1 infection, such as presence of antibodies to virus proteins, by routine diagnostic techniques. The virus strain itself has an altered pattern of glycosylation and/or the different oligomeric structure of Env due to altered variable loops. Consequently, the functional components of Env that are potential neutralising epitopes of Env are no longer exposed to or accessible to the immune system.

The inventors characterised Env and developed Env-immunogens from viral isolates from patients presenting with acute symptoms of HIV infection before antibodies to virus proteins were evident. The Env antigenic variations were found in transmission strains. Cloned transmission strain Env expressed in cells as gp160 precursors that process into gp120 and gp41 oligomers function to permit binding and fusion with cells expressing the CD4 surface receptor together with the CCR5 coreceptor, and in some embodiments both the CCR5 and the CXCR4 coreceptors. Transmission strain Env may contribute to the selective entry of virus at the mucosal surface and subsequent evolution of Env serves to conceal the functional components of Env and evade the maturing host immune responses. This invention is therefore broadly directed to these structural differences and neutralisation sensitive epitopes suitable for eliciting Nabs, and neutralising monoclonal antibodies made therefrom.

In one aspect of the invention, there is provided a polypeptide being all or a fragment of an HIV-1 envelope protein from a transmission strain of HIV-1, the polypeptide having neutralisation sensitive epitopes that are accessible. The polypeptide is isolated from its natural environment and in turn may be used in compositions and methods of the invention.

The polypeptide may be a full length version of the envelope polypeptide from the virus around 840 to 860 amino acids in length or a fragment of an HIV-1 envelope polypeptide from a transmission strain of HIV-1. The fragment must be of sufficient length to form an oligomeric structure, preferably a trimer dimer or dimer trimer spike. Preferably the fragment is about 100 to 1000 amino acids in length, more preferably about 200 to 850 amino acids in length, and most preferably about 450 to 700 amino acids in length.

In a preferred embodiment, the polypeptide, being all or a fragment of an HIV-1 envelope protein from a transmission strain of HIV-1, has an altered glycosylation profile compared to a glycosylation profile of an envelope protein from post-seroconversion strains of HIV-1. The fragments presented as soluble gp140 oligomers function for CD4 binding and bind reference monoclonal Nabs, including those whose reactivity is induced after CD4-binding.

By “glycosylation profile” it is meant the number and/or location of potential glycosylation sites in an envelope amino acid sequence, as well as the degree of glycosylation of the polypeptide. By “altered glycosylation profile” it is meant that the glycosylation profile of the polypeptide or fragment from a transmission strain of HIV-1 is (i) different from the glycosylation profile of sequences of envelope polypeptides of blood-derived post-seroconversion HIV-1 strains which have essentially the same function or (ii) has sequence differences from consensus envelope sequences of post-seroconversion HIV-1 strains while still sharing at least 95% sequence homology. The alteration of glycosylation profiles may be determined by, for example, comparison of the polypeptide sequence with a consensus envelope polypeptide of the same strain of HIV-1 isolated from a patient who has already seroconverted and cleared HIV strains that have Env that is sensitive to Nab. The exact location of individual glycosylation sites can vary slightly depending on the strain of HIV-1. In one embodiment alteration of the glycosylation profile of subtype B HIV envelope polypeptide may be determined by comparison with the consensus sequence of the envelope polypeptide of HIV_{HXB2} of accession number K03455 version K03455.1 dated 21 Oct. 2002. The envelope region from 6225 to 8795 is also provided in SEQ ID NO: 35; the corresponding gp160 envelope amino acid sequence is provided in SEQ ID NO: 36.

An altered glycosylation profile can result from deletion, modification, or mutation of potential glycosylation sites within the HIV envelope polypeptide, particularly N-linked glycosylation sites. By “accessbility” and “modulation of accessibility” it is meant that the location of particular glycosylation sites and the glycosylation profile thereafter results in neutralisation sensitive epitopes on the mature oligomer being exposed to the immune system whereas they would otherwise be masked by glycans. In other words, in the absence of glycosylation, neutralising epitopes on the functional oligomer that may otherwise be obscured by glycans are exposed or accessible to the immune system. Alternatively, altered glycosylation profiles may have one or more additional glycosylation sites or lost glycosylation sites that result in different Env folding and subsequently exposure and/or accessibility of functional domains that are susceptible to neutralising antibody. Accordingly, in one embodiment of the invention, the glycosylation profile of the polypeptide is altered as a result of deletion, modification, or mutation of one or more glycosylation sites within the HIV-1 envelope polypeptide.

The HIV envelope polypeptide is composed of non covalently linked monomers of gp120 that assemble into a trimeric unit (approximately 500 amino acids depending on the isolate or strain of HIV-1) and gp41 (approximately 350 amino acids depending on the isolate or strain of HIV-1). The one or more glycosylation sites may be in either gp120 or gp41, or both. The surface of gp120 consists of a single peptide having 5 conserved domains (C1-C5) and 5 variable domains (V1-V5) [FIG. 1]. Accordingly, when the one or more glycosylation sites are in gp120, they may be in the variable domains, the conserved domains, or both. Preferably the glycosylation sites are in the C4 domain, or one or more of the V1-V5 domains.

N-linked carbohydrates are added at glycosylation sites that are typically located at an Asparagine residue within the consensus amino acid sequence motif N-X-S, or N-X-T (where X is any amino acid). In one embodiment, the altered glycosylation profile results from changes to one or more glycosylation sites in conserved regions. In another embodiment, the one or more glycosylation sites are in the variable domains V1-V5 of gp120, particularly V3 and V4.

In a particularly preferred embodiment the mutated or deleted glycosylation site is one or more of:

| Site | Env |
|---|---|
| 136 | V1 |
| 141 | V1 |
| 160 | V2 |
| 197 | C2 |
| 230 | C2 |
| 234 | C2 |
| 289 | C2 |
| 295 | C2/V3 |
| 339 | C3 |
| 358 | C3 |
| 386 | V4 |
| 637 | Ectodomain of gp41 |
| 674 | Ectodomain of gp41 |

The number is with reference to the HIV-1 strain HXB2 (SEQ ID NO: 36 based on accession number: K03455 version K03455.1). The skilled person will appreciate that the location of these N-linked glycosylation sites from the consensus motif (N-X-S, or N-X-T) will vary with different isolates and strains of HIV, and that based on sequence alignments and other known methods in the art, it is possible for the skilled person to identify the analogous sites in other strains and isolates.

As mentioned above, the inventors characterised transmission strains of HIV-1 and other strains of HIV-1 that evolved without neutralising-antibody selection as having altered variable loops with the Env polypeptide. Accordingly, in another aspect of the invention, there is provided a polypeptide being all or a fragment of an HIV-1 envelope protein from a transmission strain of HIV-1, the polypeptide having neutralisation sensitive epitopes that are accessible, wherein the envelope protein from the transmission strain has one or more altered variable loop sequences compared to variable loop sequences of an envelope protein from post-seroconversion strains of HIV-1.

In a preferred embodiment, the V4 loop is shortened compared with the reference isolate, HXB2 (SEQ ID NO: 36 based on accession number: K03455 version K03455.1).

Variable loop sequences by their nature exhibit some variation from isolate to isolate, and strain to strain. The invention, however, describes natural Env gp160 variants with altered glycosylation profiles that are selected during mucosal transmission but rarely found in blood after seroconversion due to high susceptibility to neutralising antibody. When presented as gp160 trimers, or gp140 trimers they prominently expose elaborate structures on the trimers that are important targets for neutralising antibodies, and themselves serve as immunogens that can stimulate the production of neutralising antibodies. The transmission strain Env in both gp160 and gp140 forms bind CD4 and adopt a second conformation that further reveals binding sites for CCR5 coreceptor in one embodiment, or both CCR5 and CXCR4 in another embodiment. After binding to CD4 some embodiments of the soluble gp140 also further display structural conformations that increase the affinity of binding monoclonal Nab targeting conserved CD4-induced epitopes. These findings demonstrate that soluble gp140 trimers of transmission strain Env adopt prominently display conserved functional components of the Env structure.

Accordingly, by “altered” it is meant that that the variable loop sequences are longer or preferably shorter when compared with other HIV envelope polypeptide sequences derived from post-seroconversion isolates having the same function, or HIV envelope polypeptide sequences derived from post-seroconversion isolates having at least 95% sequence identity. As a result of the altered variable loop sequences, neutralising sensitive epitopes are exposed and/or accessible. In the oligomeric envelope structure of non-altered variable loops, the epitopes would be obscured due to the 3-dimensional folding. Alternatively, if not obscured or inaccessible, the epitopes may be sufficiently conformationally different to avoid Nab binding.

The variable domain consisting of variable loops V1-V5 may be altered overall in length. i.e., the V1-V5 domain is shorter or longer than the variable domain of an HIV envelope polypeptide sequence having the same function or having at least 95% sequence identity.

In another embodiment, the overall length of the V1-V5 domain may not be altered but one, two, three, four or five variable loop structures may be altered. For example, V4 may be shortened, whilst V1, V2 and V5 may be lengthened.

In particular embodiments the polypeptide from a transmission strain of HIV-1 and having neutralisation sensitive epitopes that are accessible has both an altered glycosylation profile and one or more altered variable loop sequences compared to a glycosylation profile and variable loop sequences of an envelope protein from post-seroconversion strains of HIV-1 circulating in blood.

In another aspect of the invention there is provided an oligomeric polypeptide comprising a polypeptide of the invention wherein the oligomeric polypeptide generates neutralising antibodies to transmission strains of HIV-1. In one embodiment, the oligomeric polypeptide comprises a polypeptide being all or a fragment of an HIV-1 envelope protein from a transmission strain of HIV-1, wherein the envelope protein lacks a hydrophobic transmembrane anchored region. These polypeptides assemble as oligomers, such as a trimer, that presents neutralisation sensitive epitopes that are accessible. Preferably the polypeptide from a transmission strain of HIV-1 and having neutralisation sensitive epitopes that are accessible has either or both an altered glycosylation profile and one or more altered variable loop sequences of an envelope protein from post-seroconversion strains of HIV-1.

The oligomeric polypeptide can form dimer or preferably trimers. In one embodiment the furin cleavage site between gp120 and gp41 can be mutated to “RETG” to block furin cleavage during cellular expression. Furthermore, a translation termination signal around position 683 immediately prior to the transmembrane domain can be introduced, resulting in a soluble gp140 version of the Env that assembles as functional structured trimers during cellular expression.

Receptor binding regions of gp120 are considered to be key neutralising epitopes. However, previous attempts to induce broadly neutralising antibodies in humans using envelope gp120 protein as an antigen have failed. The pre-seroconversion transmission strain Env embodied in either the native gp120 and gp41 membrane bound trimers or in the soluble gp140 trimer demonstrate functional properties for receptor and co-receptor binding that are neutralising antibody targets. They also bind with monoclonal Nab with high affinity. Having determined that transmission strains of HIV have an altered glycosylation profile with fewer epitopes being “masked” by glycans, and that transmission strains may also have epitopes exposed due to a different Env conformation as a result of altered variable loops, the inventors used these epitopes to elicit neutralising antibodies.

Accordingly, the invention also provides an HIV-1 neutralising antibody for binding neutralisation sensitive epitopes, wherein the neutralisation sensitive epitopes are accessible in a transmission strain of HIV-1. Preferably the polypeptide has either or both an altered glycosylation profile and an altered variable loop sequence.

The neutralising epitopes involve the CD4 binding sites together with the surrounding amino acid and glycosylation structure. These sites may be further exposed after interacting with functional CD4 molecules.

The antibody may be monoclonal or polyclonal, preferably monoclonal. The antibody may be produced by hybridoma. Monoclonal and polyclonal antisera can be obtained by immunising a host with a polypeptide of the invention together with an adjuvant according to standard techniques.

The antibody may be a whole antibody of any isotype. Where the antibody is an antibody fragment, the antibody fragment is selected from the group consisting of a dAb, Fab, Fd, Fv, F(ab′)2, scFv and CDR.

Preferably, the antibodies of the invention are specific for neutralising sensitive HIV strains having neutralisation epitopes in the variable or constant domains of the gp120 envelope glycoprotein. The neutralisation epitopes may preferably be located in the C2 domain, C4 domain, V3 domain or the V4 domain. Methods for producing antibodies, particularly monoclonal antibodies, are well known in the art.

Neutralisation sensitive HIV-1 variants are thought to be preferentially transmitted. Accordingly, in another aspect of the invention, there is a method of reducing the risk of HIV infection in a seronegative individual, including administering to the individual an HIV-1 envelope polypeptide or polypeptide fragment wherein one or more glycosylation sites in gp120, gp41, or both are deleted or mutated. Preferably the HIV-1 envelope polypeptide or polypeptide fragment is oligomeric, having dimeric, trimeric or multiple valency forms thereof, such that the oligomeric form contains the neutralisation sensitive epitopes in an accessible arrangement.

By “seronegative” it is meant that the individual does not have any serological evidence of HIV-1 infection, such as presence of antibodies or virus, by routine diagnostic techniques.

In another embodiment there is a method of reducing the risk of HIV infection in a seronegative individual, including administering to the individual a polypeptide being all or a fragment of an HIV-1 envelope protein from a transmission strain of HIV-1, the polypeptide having neutralisation sensitive epitopes that are accessible. Preferably, the transmission strain has either or both an altered glycosylation profile and one or more altered variable loop sequences compared to a glycosylation profile and variable loop sequences of an envelope protein from post-seroconversion strains of HIV-1.

The invention also provides a method of generating an immune response to an HIV-1 envelope polypeptide in a seronegative individual, including administering to the individual a polypeptide being all or a fragment of an HIV-1 envelope protein from a transmission strain of HIV-1, the polypeptide having neutralisation sensitive epitopes that are accessible. Preferably, the envelope protein from the transmission strain has an altered glycosylation profile compared to a glycosylation profile of the envelope protein from post-seroconversion strains of HIV-1. Alternatively, or in addition to, the envelope protein from the transmission strain has one or more altered variable loop sequences compared to variable loop sequences of an envelope protein from post-seroconversion strains of HIV-1. The variable loops are preferably shortened.

In a further preferred embodiment, the polypeptide is oligomeric, having dimeric, trimeric or multiple valency forms thereof.

In one embodiment, the individual is assessed as having a suspected exposure to HIV-1, and is at risk of having being infected with HIV-1.

In another embodiment of this aspect of the invention, the individual may be administered a nucleotide sequence which encodes the polypeptide from the transmission strain of HIV-1. Such nucleotides are preferably administered in a form that allows expression of a polypeptide or fragment as a dimer, timer or multiple thereof, such as via an expression vector. As noted above, the Env expressed for generating an immune response preferably retains the oligomeric structure that contains the neutralisation epitopes. Alternatively, the individual may benefit from the administration of Env polypeptides according to the invention together with the nucleotide sequences of the invention. Administration of the nucleotide sequence may serve to prime the immune system. Administration of the polypeptide then serves to boost the immune response.

An Env polypeptide that is suitable to generate an immune response is an Env polypeptide having at least 95%, 96%, 97%, 98%, 99% or 100% amino acid identity to gp140. gp140 contains a fragment of a gp120 from a given HIV strain and a fragment of gp41 from the same HIV strain, wherein the gp41 fragment lacks the transmembrane domain (see Examples). gp140 polypeptides capable of forming oligomeric structures may be expressed from a construct. Accordingly, there is provided a nucleotide sequence that encodes for a gp140 polypeptide, and an expression construct comprising a nucleotide sequence that codes for a gp140 polypeptide. The expression construct may be one for transient use or be more suitable for stable transfection and maintenance within a target cell as either an episomally replicating construct or an integrated form.

In another embodiment, the Env polypeptide that is suitable to generate an immune response is an Env polypeptide having at least 95%, 96%, 97%, 98%, 99% or 100% amino acid identity to gp120.

In one embodiment there is provided a gp140 polypeptide, and an expression construct that expresses a gp140 polypeptide. In another embodiment, there is provided a glycosylated gp140 polypeptide, which has been manufactured or expressed in an expression system that adds the native cellular glycosylation.

Accordingly, methods for reducing the risk of HIV infection in a seronegative individual may also involve the administration of an expression construct for expressing a nucleotide sequence that encodes for the polypeptides of the invention, the polypeptides preferably having either or both of an altered glycosylation profile, or an altered variable loop sequence. Preferably the nucleotide sequence encodes the Env polypeptide gp160 that matures into gp120 and gp41, or the gp140 versions of these polypeptides.

In an alternative embodiment, the expression construct may express the polypeptides of the invention, the polypeptides preferably having either or both of an altered glycosylation profile, or an altered variable loop sequence wherein the envelope polypeptide or polypeptide fragment is capable of forming an oligomeric structure. Preferably the Env polypeptide expressed is gp140.

In another aspect of the invention there is provided a composition for use in raising an immune response comprising an HIV-1 envelope polypeptide or polypeptide fragment wherein one or more glycosylation sites in gp120, gp41, or both are deleted or mutated.

There is also provided, a composition for use in raising an immune response against HIV-1, the composition comprising a polypeptide being all or a fragment of an HIV-1 envelope protein from a transmission strain of HIV-1, the polypeptide having neutralisation sensitive epitopes that are accessible. In one embodiment, the envelope protein from the transmission strain has an altered glycosylation profile compared to a glycosylation profile of the envelope protein from post-seroconversion strains of HIV-1.

In an alternative embodiment, the envelope protein from the transmission strain has one or more altered variable loop sequences compared to variable loop sequences of an envelope protein from post-seroconversion strains of HIV-1.

In yet another embodiment, the envelope protein from the transmission strain has both an altered glycosylation profile and one or more altered variable loop sequences compared to a glycosylation profile and variable loop sequences of an envelope protein from post-seroconversion strains of HIV-1.

Preferably the immune response is generated against a transmission or pre-seroconversion strain of HIV-1.

In an alternative embodiment, the composition may comprise the neutralising antibodies of the invention themselves that have been raised against an HIV envelope polypeptide having an altered glycosylation profile and/or altered variable loop sequence.

The compositions of the invention may be suitable as pharmaceutical compositions and further include a pharmaceutically acceptable carrier, diluent, excipient or like compound. By “pharmaceutically acceptable” it is meant that the substance or composition must be compatible chemically and/or toxicologically, with the other ingredients comprising a formulation, and/or the mammal being treated therewith.

Acceptable diluents, carriers, excipients, and stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as plasma albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The composition may be prepared for various routes and types of administration.

The pharmaceutical composition may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

In an alternative embodiment, there is provided a method of reducing the risk of HIV infection including administering an HIV-1 neutralising antibody for binding neutralisation sensitive epitopes, wherein the neutralisation sensitive epitopes are accessible in a transmission strain of HIV-1. The neutralisation epitopes may be located in one or more of the C2 domain, the C4 domain, V3 domain or V4 domain of the HIV envelope polypeptide. Such a method may be particularly important for individuals who have had an acute exposure to HIV. For example, a needle stick injury. Preferably the antibody is administered in combination with other treatment regimes such as antiretroviral drugs and chemotherapeutics.

Alternatively, the individual may benefit from the administration of both a polypeptide of the invention or nucleotide sequence for expressing the polypeptide of the invention, either by themselves or from an administered expression construct and neutralising antibodies.

The term “reducing the risk” refers to prophylactic or preventative measures for protecting or precluding an individual not being infected with HIV-1 from acquiring infection with HIV-1 upon exposure to the virus.

As noted above, the neutralising antibody, polypeptide, nucleotide sequence or expression constructs may all be formulated for administration as a pharmaceutical composition. The pharmaceutical compositions of the invention are to be administered in a therapeutically effective amount. The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein. As a result of host cell responses, transmission strains of HIV-1 are selectively forced to mutate to avoid immune detection, particularly as the transmission strains have been found to be neutralisation sensitive. Despite this, HIV-1 transmission strains can persist in immunologically protected sites such as the brain, where concentrations of neutralising antibodies are reduced. Thus, it is possible that neutralisation sensitive strains of HIV-1, with altered glycosylation profiles and/or altered variable loop sequences may persist at late stages of HIV-1 infection and have augmented ability to be cytopathic in vivo if the humoral immune response is impaired.

There is therefore a need to provide a method of treatment for individuals already infected with HIV-1 particularly for those individuals yet to mount an antibody response or those already with impaired immune response.

To this effect, the present invention also provides a method of treating HIV-1 seropositive individuals to minimise symptoms of HIV-1 infection including administering to the individual a polypeptide being all or a fragment of an HIV-1 envelope protein from a transmission strain of HIV-1, the polypeptide having neutralisation sensitive epitopes that are accessible. In one embodiment, the envelope protein from the transmission strain has an altered glycosylation profile compared to a glycosylation profile of the envelope protein from post-seroconversion strains of HIV-1.

In an alternative embodiment, the envelope protein from the transmission strain has one or more altered variable loop sequences compared to variable loop sequences of an envelope protein from post-seroconversion strains of HIV-1.

In yet another embodiment, the envelope protein from the transmission strain has both an altered glycosylation profile and one or more altered variable loop sequences compared to a glycosylation profile and variable loop sequences of an envelope protein from post-seroconversion strains of HIV-1.

Preferably the polypeptide described in the embodiments above is in a form suitable to induce an immune response and retains the oligomeric structure that contains the neutralisation epitopes in an accessible conformation.

The HIV-1 envelope polypeptide administered to the individual may be a fragment of an envelope glycoprotein as described in more detail earlier.

In yet another embodiment of this aspect of the invention, the individual may be administered a nucleotide sequence which encodes an envelope protein from a transmission strain of HIV-1. Such nucleotides are preferably administered in a form that allows expression of the envelope polypeptide as a dimer, trimer or multiple valency thereof such as via an expression vector.

Treatment of HIV-1 seropositive individuals with polypeptides of the invention, nucleotides or nucleotide fragments of the invention, expression vectors for expressing either the polypeptide or nucleotide of the invention, or a neutralising antibody of the invention may be combined with other treatment regimes. Examples of chemotherapeutics that are useful for treatment of HIV-1 include lamivudine, emtricitabine, abacavir, zalcitabine, dideoxycytidine, azidothymidine, tenofovir, disoproxil fumarate, didanosine, dideoxyinosine, stavudine, delavirdine, efavirenz, nevirapine, amprenavir, tipranavir, indinarvir, saquinavir, saquinavir mesylate, lopinavir, ritonavir, fosamprenavir calcium, darunavir, atazanavir sulfate, enfuvirtide, and nelfinavir mesylate.

Alternatively, the seropositive individual may benefit from the administration of both the polypeptide and neutralising antibodies of the invention. Administration may by concurrent or sequential. Administration of the polypeptide may be via expression from an expression construct.

There is also provided use of a composition in the manufacture of a medicament for reducing the risk of HIV infection comprising as an active ingredient a polypeptide being all or a fragment of an HIV-1 envelope protein from a transmission strain of HIV-1, the polypeptide having neutralisation sensitive epitopes that are accessible. The envelope protein from the transmission strain may have either or both an altered glycosylation profile and one or more altered variable loop sequences compared to a glycosylation profile and variable loop sequences of an envelope protein from post-seroconversion strains of HIV-1.

The compositions used in the manufacture of a medicament may also comprise as the active ingredient:
a nucleotide sequence which encodes a polypeptide being all or a fragment of an HIV-1 envelope protein from a transmission strain of HIV-1, the polypeptide having neutralisation sensitive epitopes that are accessible;
an HIV-1 neutralising antibody, preferably a monoclonal antibody, for binding neutralisation sensitive epitopes, wherein the neutralisation sensitive epitopes are accessible in a transmission strain of HIV-1;
an oligomeric polypeptide comprising a polypeptide being all or a fragment of an HIV-1 envelope protein from a transmission strain of HIV-1, the oligomeric polypeptide having neutralisation sensitive epitopes that are accessible; or
an expression construct that express a nucleotide sequence which encodes a polypeptide being all or a fragment of an HIV-1 envelope protein from a transmission strain of HIV-1, the polypeptide having neutralisation sensitive epitopes that are accessible, or encodes the polypeptide itself.

Preferably the polypeptide of the invention described in the embodiments above is in a form suitable to induce an immune response and retains the oligomeric structure that contains the neutralisation epitopes.

In a further embodiment of the invention, there is provided a kit for preventing or treating an individual. The kit includes one or more of:
a polypeptide being all or a fragment of an HIV-1 envelope protein from a transmission strain of HIV-1, the polypeptide having neutralisation sensitive epitopes that are accessible;
an HIV-1 neutralising antibody, preferably a monoclonal antibody, for binding neutralisation sensitive epitopes, wherein the neutralisation sensitive epitopes are accessible in a transmission strain of HIV-1;
a nucleotide sequence which encodes a polypeptide being all or a fragment of an HIV-1 envelope protein from a transmission strain of HIV-1, the polypeptide having neutralisation sensitive epitopes that are accessible;
an oligomeric polypeptide comprising a polypeptide being all or a fragment of an HIV-1 envelope protein from a transmission strain of HIV-1, the polypeptide having neutralisation sensitive epitopes that are accessible;
   and optionally a package insert for use of the polypeptide, or the antibody, or the nucleotide sequence, or a combination thereof for treating HIV-1 seropositive individuals to reduce the risk of HIV-1 or minimise symptoms of HIV-1 infection.

In an alternative embodiment the kit is used for reducing the risk of HIV-1 infection.

The term “package insert” is used to refer to instructions customarily included in commercial packages of therapeutic products, that contain information about the indications, usage, dosage, administration, contraindications and/or warnings concerning the use of such therapeutic products.

### BRIEF DESCRIPTION OF THE DRAWINGS

FIG. 1 is a schematic of the Envelope gp160 polypeptide of HIV illustrating the relative location of the variable (V) and constant (C) domains in gp120 as well as the domains within gp41.

FIG. 2 illustrates the cloning process from the gp160 clones to produce the gp140 expression clones for expressing soluble gp140.

FIG. 3A-3D is a sequence alignment of select gp160 clones.

FIG. 4 is a western blot analysis of the expression of soluble gp140 from each of the clones. SN is the gp140 detected from supernatant samples (ie extracellular) and CL is the gp140 detected from cell lysates (ie intracellular). Expression was compared with an HIV_{AD8} control (pNLAD8) as a positive control, and empty vector (pN1 EGFP) as a negative control.

FIG. 5 demonstrates the ability of the pre-seroconversion transmission strains HIV Env gp140 in SC24, 35, 73 and 89 to bind to four domain soluble CD4. SC182 is a non-fucntinal control.

FIG. 6 shows the results of enzyme linked immuno-sorbant assays (ELISA) that show the binding affinity of monoclonal neutralising antibodies (mNab) known to neutralise many strains of HIV-1 to the pre-seroconversion transmission strains HIV Env gp140 from SC24, 35, 73 and 89. The binding of these mNabs define epitopes that are common neutralisation targets that are shared by most HIV-1 strains. NC=negative control−293T and HeLa cell proteins; the positive controls were AD8 and pNL4-3. SC182 is a non-functional gp140 control.

FIG. 7A-B illustrate the in vitro neutralisation assay results from the vaccination of groups of mice with constructs containing gp140 encoding sequences from transmission strains of HIV-1, and protein boost formulations of oligomeric gp120 in the form of uncleaved gp140 protein from the same transmission strains respectively of HIV-1. The ability of the antibodies generated in the vaccinated mice to neutralise pseudotype EGFP reporter viruses AD8 (an R-5 tropic strain of HIV-1—FIG. 7A), and homologous viruses (AD8, SC24, SC35 and SC73—FIG. 7B) infectivity of target cells in an Env dependent manner was evaluated.

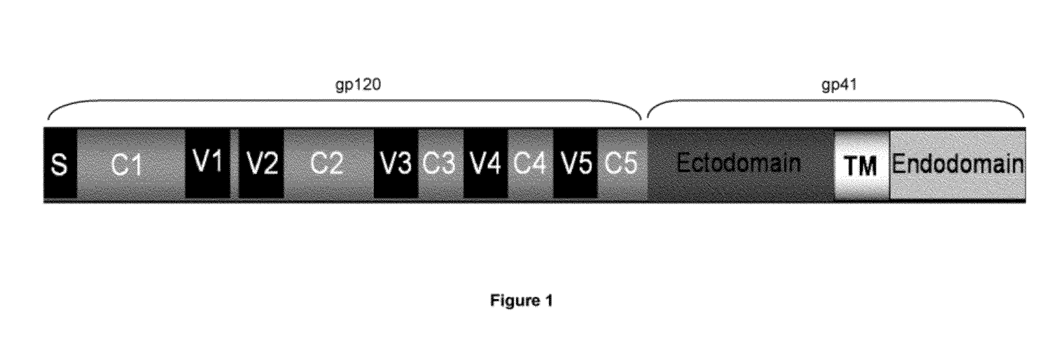

### EXAMPLES

It will be understood that the invention disclosed and defined in this specification extends to all alternative combinations of two or more of the individual features mentioned or evident from the text or drawings. All of these different combinations constitute various alternative aspects of the invention.

### Example 1

### Sample Selection

Patients presenting with acute symptoms of HIV-1 infection enrolled under different clinical studies were grouped to form a pre-seroconversion cohort on the basis of preliminary screening results i.e. positive/negative proviral DNA, viral load, p24 antibody titers, negative/positive consecutive ELISA and negative/indeterminate western blot (no or ⅔ HIV-1 specific bands).

From this cohort, 38 samples were for further investigation based on high viral load, high p24 antigen titers, negative/positive ELISA and negative western blot. Sequencing of the 38 samples identified 5 patients with viral isolates exhibiting envelope polypeptide changes and who were found to fall within stage 1V of early infection i.e. possibly within 30 days following infection.

### Example 2

### Cloning and Sequence Analysis

RNA samples were reverse transcribed to cDNA followed by a nested PCR approach to generate near full length HIV-1 gp160 envelope fragments. The fragments were cloned between KpnI and BamHI restriction sites in pSVIIIΔks vector. Due to potential envelope toxicity the clones were isolated by growing at a lower temperature of 30° C. In total 28 clones were isolated across 5 samples.

All 28 gp160 clones were sequenced from mini/maxi preparations of DNA clones using Big-Dye terminator sequencing method. DNA and protein sequences were aligned and analysed using Vector NTI program. N-glycosylation sites were analysed using N-Glycosite tool (HIV sequence database). On analysis of DNA and protein sequences, these clones were found to have significant differences in variable loop length, amino acid chain length (Table 1) and reduced N-linked glycosylation sites in comparison to reference strains pNL4.3 (Genbank accession no. M19921) (T cell tropic—SEQ ID NO: 39) and AD8 (M cell tropic—SEQ ID NO:41) (Table 2). A summary of the particular glycosylation sites altered is provided in Table 3.

The clones and constructs may be referred to interchangeably throughout the specification as, for example, “SC-24” or “PSC-24”.

TABLE 1
Summary of the number of amino acids in each variable domain
of the gp160 clones, as well as total number of amino acids
for the V1-V4, V1-V5, gp120, gp41 and gp160 regions.

| | Samples | | | | | | | | | |
|---|---|---|---|---|---|---|---|---|---|---|
| | | | | | | V1- | V1- | | | |
| | V1 | V2 | V3 | V4 | V5 | V4 | V5 | gp120 | gp41 | gp160 |
| pNL4.3 | 27 | 38 | 36 | 34 | 9 | 286 | 339 | 509 | 345 | 854 |
| AD8 | 24 | 40 | 35 | 35 | 10 | 285 | 339 | 508 | 346 | 854 |
| SC 24 | 29 | 41 | 35 | 29 | 11 | 284 | 339 | 509 | 345 | 854 |
| SC 35 | 34 | 43 | 28 | 31 | 8 | 291 | 343 | 513 | 345 | 858 |
| SC 73 | 29 | 41 | 35 | 29 | 11 | 284 | 339 | 509 | 345 | 854 |
| SC 89 | 25 | 42 | 35 | 31 | 12 | 283 | 339 | 509 | 346 | 855 |
| SC 182 | 25 | 42 | 35 | 31 | 12 | 283 | 339 | 509 | 346 | 855 |

TABLE 2
Summary of average number of potential glycosylation
sites identified in each of the gp160 clones for each
variable domain of Env, as well as total number of
sites for the V1-V4, V1-V5 and gp120 regions.

| | Samples | | | | | | | | | |
|---|---|---|---|---|---|---|---|---|---|---|
| | | | | | | V1- | V1- | | | |
| | V1 | V2 | V3 | V4 | V5 | V4 | V5 | gp120 | Ecto | gp41 |
| pNL4.3 | 3 | 2 | 1 | 4 | 1 | 21 | 23 | 24 | 5 | 7 |
| AD8 | 4 | 2 | 1 | 6 | 1 | 23 | 25 | 27 | 4 | 5 |
| SC 24 | 4 | 2 | 1 | 3 | 2 | 19 | 22 | 25 | 3 | 4 |
| SC 35 | 4 | 2 | 0 | 4 | 1 | 19 | 21 | 23 | 4 | 6 |
| SC 73 | 4 | 2 | 1 | 3 | 2 | 19 | 22 | 25 | 3 | 4 |
| SC 89 | 2 | 2 | 1 | 5 | 2 | 18 | 21 | 24 | 4 | 6 |
| SC 182 | 2 | 2 | 1 | 5 | 2 | 18 | 21 | 24 | 4 | 6 |

TABLE 3
Summary of the particular glycosylation sites
altered within gp160 clones.

| Sample/Clone Numbers | Altered N-Glycosylation sites | Total |
|---|---|---|
| SC 24 - 76 & 94 | 230, 289, 339, 386, 637, 674 | 6 |
| SC 35 - 5 & 10 | 230, 234, 295, 339, 386, 674 | 6 |
| SC 73 - 47, 48 78 | 230, 289, 339, 386, 637, 674 | 6 |
| SC 73 - 271 | 141, 160, 386, 674 | 4 |
| SC 89 - 21 & 51 | 136, 230, 234, 295, 674 | 5 |
| SC 182 - 156 | 136, 230, 234, 295, 674 | 5 |

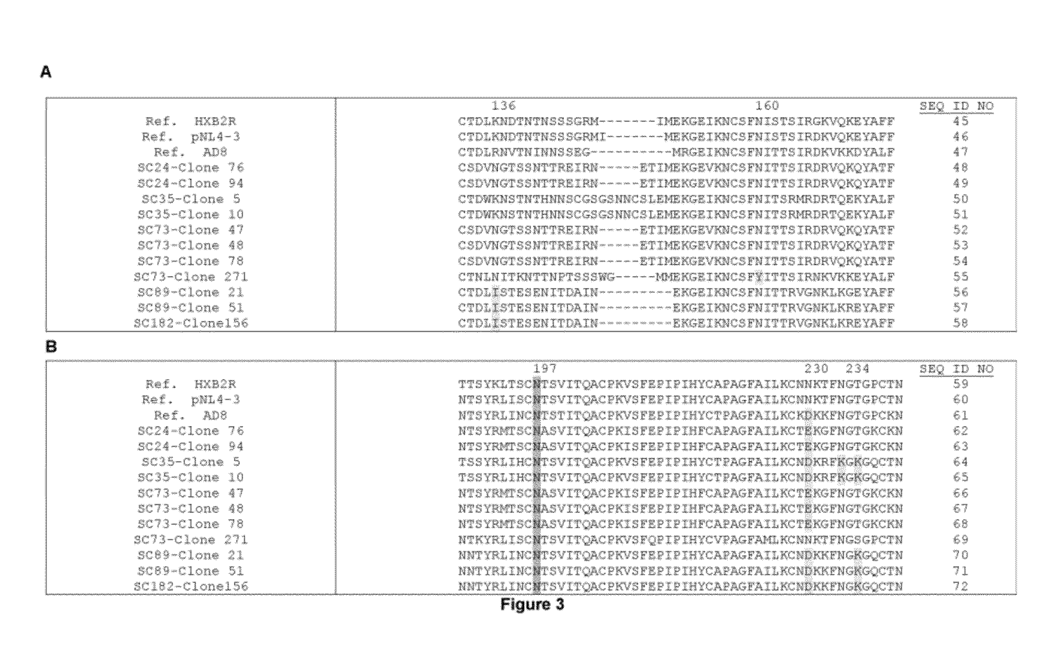

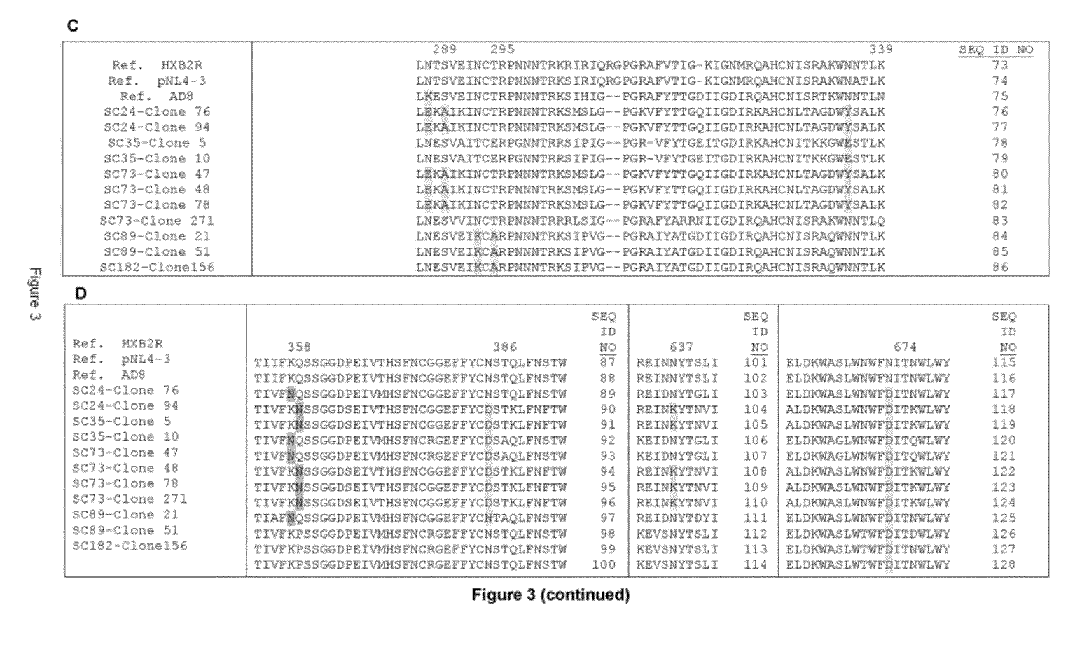

Alignment of each clone in Table 3 with the gp160 sequence of laboratory derived strains HXB2 (SEQ ID NO: 36), pNL4.3 (SEQ ID NO:39) and AD8 (SEQ ID NO: 41) is shown in FIG. 3A-3D. Dark grey boxes identify unaltered glycosylation sites; light grey boxes identify altered glycosylation sites.

### Example 3

### Functional Assay and Designing Immunogens

All 28 gp160 clones were tested for envelope mediated entry by pseudotyping a GFP reporter virus to assay infectivity in permissive JC 53 cells expressing CD4, CCR5 and CXCR4 receptors. The results were analysed using fluorescence microscopy and confirmed by FACS. Out of the 28 clones, 10 (36%) were found to be functional, as summarised in Table 4.

Co-receptor usage is summarised in Table 4B. The gp160 clones were tested for envelope receptor mediated entry by pseudotyping a GFP or luciferase reporter virus in a single round entry assay in cells expressing CD4, CCR5/CXCR4.

TABLE 4
Functional Assay - Results

| Samples | SC 24 | SC 35 | SC 73 | SC 89 | SC 182 | Total |
|---|---|---|---|---|---|---|
| # | 3 | 10 | 4 | 10 | 1 | 28 |
| Clones | | | | | | |
| tested | | | | | | |
| # | 2 (Clones | 2 (Clones | 4 (Clones | 2 (Clones | 0 | 10 |
| func- | 76 and 94) | 5 and 10) | 47, 48, 78, | 21 and | | |
| tional | | | 271) | 51) | | |
| % | 66.66 | 20 | 100 | 20 | 0 | 35.71 |
| Func- | | | | | | |
| tional | | | | | | |

TABLE 4B
CD4 and coreceptor usage

| | Tropism | | Fusogenicity |
|---|---|---|---|
| Samples | GFP | Luciferase | Fusion Assay |
| SC 24 | R5X4 | R5X4 | Medium |
| SC 35 | R5 | R5 | High |
| SC 73 | R5X4 | R5X4 | Medium |
| SC 89 | R5 | R5 | High |

The SEQ ID NOS for the 10 functional clones are as follows in Table 5.

TABLE 5
SEQ ID NOS for functional gp160 clones

| gp160 Clones | Nucleotide Sequence | Amino Acid Sequence |
|---|---|---|
| SC24 - Clone 76 | 1 | 2 |
| SC24 - Clone 24 | 3 | 4 |
| SC 35 - Clone 5 | 5 | 6 |
| SC35 - Clone 10 | 7 | 8 |
| SC73 - Clone 47 | 9 | 10 |
| SC73 - Clone 48 | 11 | 12 |
| SC73 - Clone 78 | 13 | 14 |
| SC73 - Clone 271 | 15 | 16 |
| SC89 - Clone 51 | 17 | 18 |
| SC89 - Clone 51 | 19 | 20 |
| SC182 - Clone 156 | 21 | 22 |

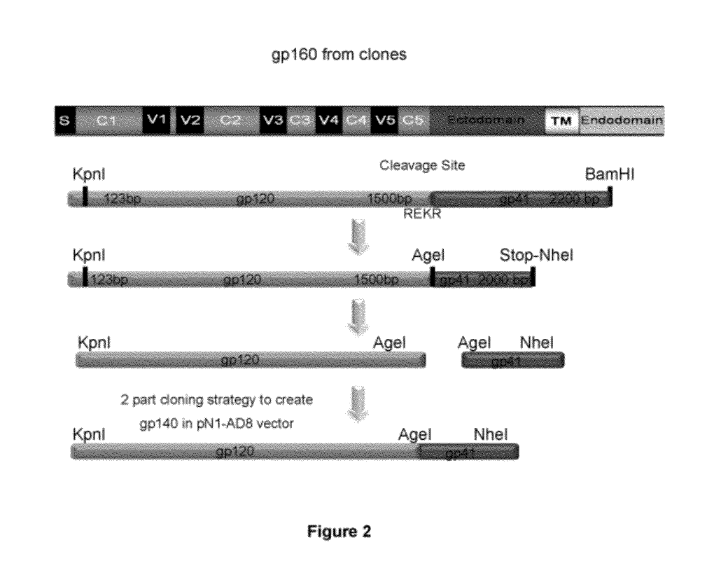

The functional gp160 clones were used to generate gp140 clones in accordance with the flow chart in FIG. 2 that express soluble Env analogues. By placing an amino acid stop codon immediately prior to the transmembrane domain, translation is terminated and allows for secretion of gp140 into the tissue culture supernatant. The amino acid cleavage motif between the gp120 and gp41 domains was changed (amino acids REKR changed to RETG) to eliminate the cleavage of gp140. Both these changes were made by in vitro mutagenesis using PCR (see further details in example 4 below).

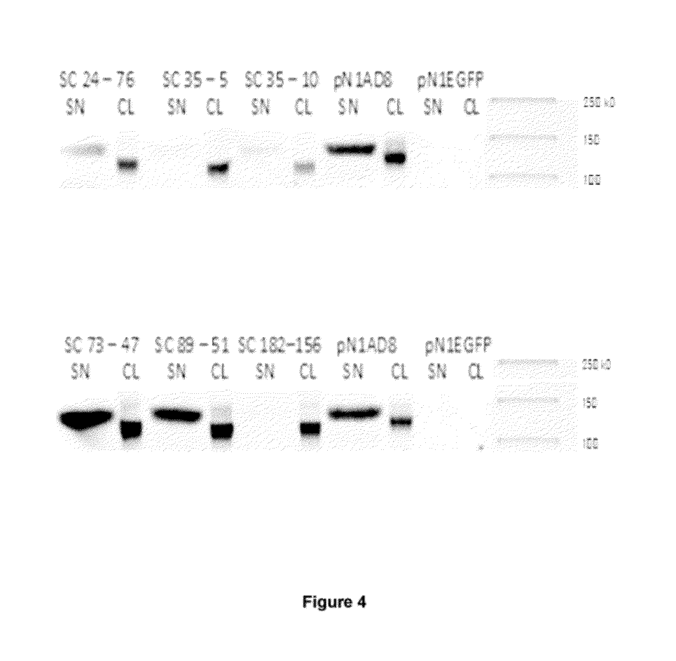

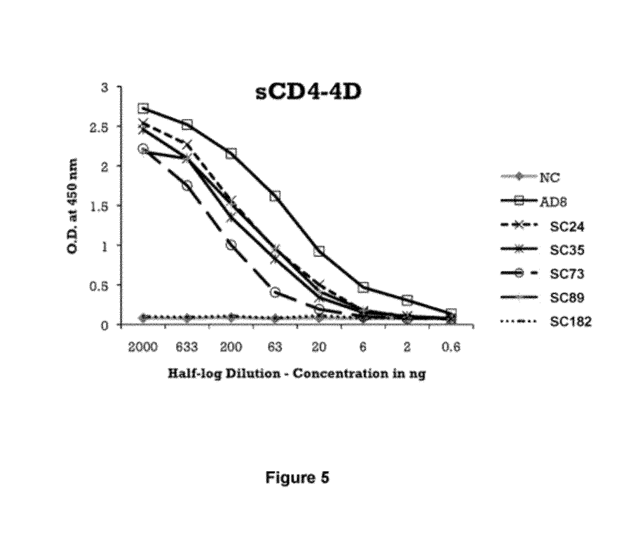

The envelope region previously cloned in pSVIIIΔks cloning vector was shuttled into a pN1 expression vector using a two-part cloning strategy wherein the gp120 fragment was cloned into the expression vector as a separate step from cloning of the gp41 fragment into the same expression vector. These changes enabled the production of stable soluble gp140. The gp140 clones were transfected into HeLa or 293T cell lines using lipofectamine. Extracellular (supernatant) and intracellular (cell lysates) expression of envelope proteins was screened for soluble gp140 expression. All 6 clones showed gp140 expression in supernatant and cell lysates in varying levels (FIG. 4). Selected gp140 clones may be used to generate stable cell lines expressing soluble gp140.

gp140 may be purified by a number of different means. For example, gp140-containing tissue culture supernatants may be passed over lentil lectin affinity columns, which mediate the capturing of glycoproteins, including gp140, through the affinity of lentil lectin for carbohydrate. After washing, gp140 is eluted competitively from the column by the addition of 0.5M Methyl -D-mannopyranoside (Sigma). Yields obtained with this system for other gp140 strains have varied between 0.4 and 1.0 milligram per 100 millilitres of tissue culture supernatant. The eluate may then be concentrated and further purified by gel filtration over superdex 200.

The gp140 is purified from the culture supernatants in an oligomeric form.

The SEQ ID NOS for the 6 selected clones showing gp140 expression in FIG. 4 are as follows in Table 6. The sequences of gp140 clones from control HIV-1 strains are SEQ ID NO: 40 for NL4.3, SEQ ID NO: 38 for HXB2 and SEQ ID NO:37 for AD8.

TABLE 6
SEQ ID NOS for functional gp140 clones

| gp140 Clone | Nucleotide Sequences | Amino Acid Sequence |
|---|---|---|
| SC24 - Clone 76 | 23 | 24 |
| SC35 - Clone 5 | 25 | 26 |
| SC35 - Clone 10 | 27 | 28 |
| SC73 - Clone 47 | 29 | 30 |
| SC89 - Clone 51 | 31 | 32 |
| SC182 - Clone 156 | 33 | 34 |

### Example 4

### Targeting Env with Well Exposed Neutralisation Targets

An effective prophylactic composition for generating an immune response to HIV-1 must be able to target newly transmitted virus to prevent HIV-1 infection from being established. As noted above, the expression constructs of the invention may be expressed from mammalian cells stably transduced with expression plasmids that use a CMV promoter, include an upstream artificial intron, fuse the major splice donor of HIV (SD1) to a splice acceptor upstream (5′) from the translation start site for Rev (SA4a, 4b, or 4c), but downstream (3′) from the translation start site for Tat (SA3/SA4), contain a short upstream open reading frame (uORF) consisting of an AUG-TAA sequence that overlaps by one nucleotide the translation start sequence (AUG) for Vpu, and a poly(A) signal sequence to terminate transcription and augment translation. The Env expression constructs co-express Vpu protein.

As detailed above, cleavage site at the junction of gp120 and gp41 is mutated and a termination signal is introduced at the junction between the gp41 ectodomain and the transmembrane domain of the of gp41 to enable expression of soluble gp140 oligomers that highly expose conserved domains required for functional receptor engagement. These oligomers have been found to bind antibodies that have broad neutralising activity, such as the b12 monoclonal NAb that binds the CD4-binding site, and when delivered as purified Env gp140 formulated into adjuvants that include incomplete Freund's adjuvant, or montanide adjuvant successfully elicit high titre antibody responses (up to 1:100,000 titre) in mouse and primate.

These antibodies display strong HIV neutralising activity for HIV-1 reporter viruses pseudotyped with a wide variety of HIV Env gp120/41. This neutralising activity against a broad array of CCR5-tropic HIV strains, including primary HIV strains that represent strains frequently existing in body secretions from HIV infected donors that lead to infection of naïve HIV recipients during transmission.

### Example 5

### The Relationship Between N197, N358, and N386 Sensitivity to Inhibition by Neutralizing Antibodies

Luciferase reporter viruses pseudotyped with a subset of the pre-seroconversion R5Envs variously lacking and/or containing combinations of N197, N358 and N386 or mutations that substitute an amino acid that will not form an active glycosylation site in were produced by transfection of 293T cells with pCMVΔP1ΔenvpA, pHIV-1Luc and pSVIII-Env or pCMV-Env plasmids using Lipofectamine 2000 (Invitrogen) at a ratio of 1:3:1. Supernatants were harvested 48 h later and filtered through 0.45 μm filters. Recombinant luciferase reporter viruses were ultracentrifuged through a 25% (vol/vol) sucrose cushion at 25,000 rpm for 2 h at 4° C. using a Beckman Ultra high speed centrifuge and a SW28 rotor, resuspended in 2 ml culture medium, aliquotted and stored at −80° C. The TCID₅₀ of virus stocks was determined by titration in JC53 cells.

The ability of human monoclonal antibodies (mAbs) against HIV-1 gp120 (IgG1b12, 2G12) and the polyclonal antibody HIV-Ig to neutralize the infectivity of Env-pseudotyped luciferase reporter viruses was assayed using JC53 cells. Two hundred TCID₅₀ of each Env-pseudotyped luciferase reporter virus (equating to an MOI of 0.02) was incubated with 10-fold increasing concentrations of each mAb (0.0005 to 50 μg/ml) or HIV-Ig (1 to 10,000 μg/ml) for 1 hr at 37° C. The virus-Ab mixtures were then used to inoculate JC53 cells overnight at 37° C. Cells were rinsed twice with culture medium to remove residual virus inoculum and incubated a further 48 h at 37° C. Virus infectivity was then measured by assaying luciferase activity in cell lysates.

The sensitivity of Env-pseudotyped luciferase reporter viruses to neutralization by Env monoclonal antibodies IgG1b12 or 2G12, or by the polyclonal antibody HIV-Ig was determined by calculation of IC₅₀ and IC₉₀ values of neutralization curves. For sensitivity to neutralization by IgG1b12 Envs with N197, N358 glycosylation sites, together with an amino acid sequence, such as a D, that eliminated the N-linked glycosylation site at 386 demonstrated high neutralisation sensitivity. The relationship between HIV-1 entry kinetics and IC₅₀ or IC₉₀ for IgG1b12 was determined by plotting the inhibitory concentrations against the maximum delay of entry. There was a positive correlation between sensitivity to neutralization by IgG1b12 and maximum delay of entry times, when either IC₅₀ or IC₉₀ of IgG1b12 were used as variables.

### Example 6

### Soluble 4-Domain CD4 Binding

Exposure of CD4 and coreceptor domains is required by HIV to establish infection and at the same time renders it susceptible to neutralization by broadly neutralizing antibodies. This is because these antibodies epitopes overlap the CD4 and coreceptor binding sites. An effective vaccine candidate should generate antibodies which can affect the binding of CD4 and coreceptors as well as neutralizing the virus. So assessing binding affinity for CD4, CD4 induced epitopes and known NAbs is essential for characterizing potential vaccine candidates.

Functional activity of pre-seroconversion transmission strain HIV Env gp140 is demonstrated through binding with high affinity to four domain soluble CD4. In these experiments four domain soluble CD4 (sCD4-4D) truncated at the transmembrane domain was purified from supernatant from a transduced 293 human cell line using Ni-NTA column, and concentrated using 10,000 NMWL column. Pure sCD4-4D (100 ng) was coated onto an ELISA plate and dilutions of transmission strain Env gp140 from SC-24, SC35, SC-73, SC-89 and SC-182 were added to determine binding after washing extensively in PBS and tween-20 detergent. The binding of Env gp140 was detected with pooled patient serum (1:1000 dilution), then anti-human horse raddish peroxidase conjugate, and tetramethylbenzidine substrate. The results presented in FIG. 5 demonstrate that the all preseroconverson Env gp120 bind to CD4 with high affinity, except for a non-functional control (SC 182).

### Example 7

### Affinity Binding to Epitopes

In addition to the analysis of the functional activity of the pre-seroconversion transmission strains Env gp140 to bind with high affinity to four domain soluble CD4, the binding affintiy of SC-24, SC35, SC-73, SC-89 and SC-182 to CD4 induced (CD41) epitopes, as well as known broady neutralizing monoclonal antibodies was determined. These data demonstrate that the pre-seroconversion Env gp140 trimers display functional epitopes involved in virus binding, entry and fusion, and epitopes that bind reference monoclonal Ab reagents and define important conserved neutralisation epitopes.

The 17b mNab binds to a CD4-induced epitope on trimeric Env that typically forms only after Env binds to CD4; the 447-52D mNab binds to a common neutralisation sensitive epitope in the V3 loop that may be altered after or by CD4 binding. The b12 mNAb binds to the CD4-binding pocket, the 2G12 mNab binds to a mannose-rich carbohydrate epitope. The 2F5 mNab identifies a common epitope in the membrane proximal region of Env.

For each binding assay, pure Env gp140 (from 100 ng) was coated onto an ELISA plate and dilutions of the appropriate mNab were added to determine binding after washing extensively in PBS and tween-20 detergent. The binding of Env gp140 was detected with horse raddish peroxidase conjugate anti-human antibody (1:1000 dilution).

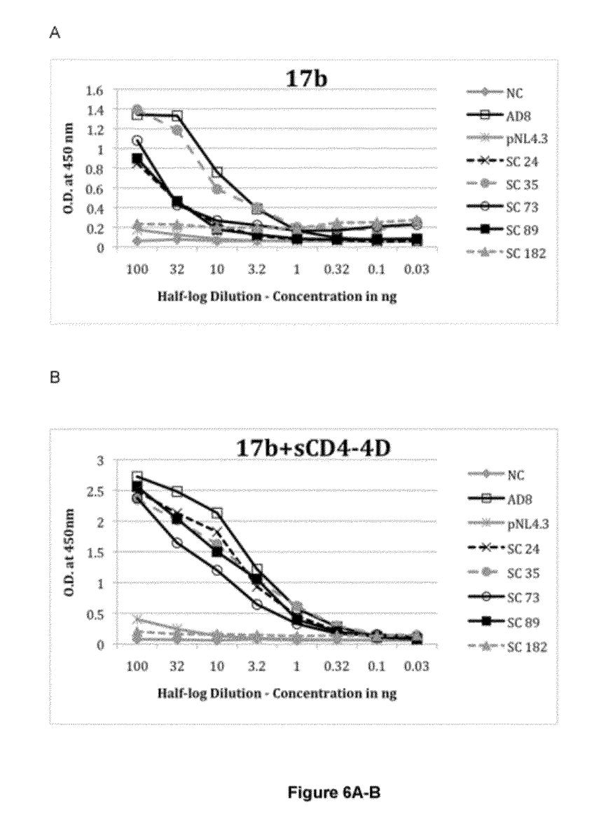

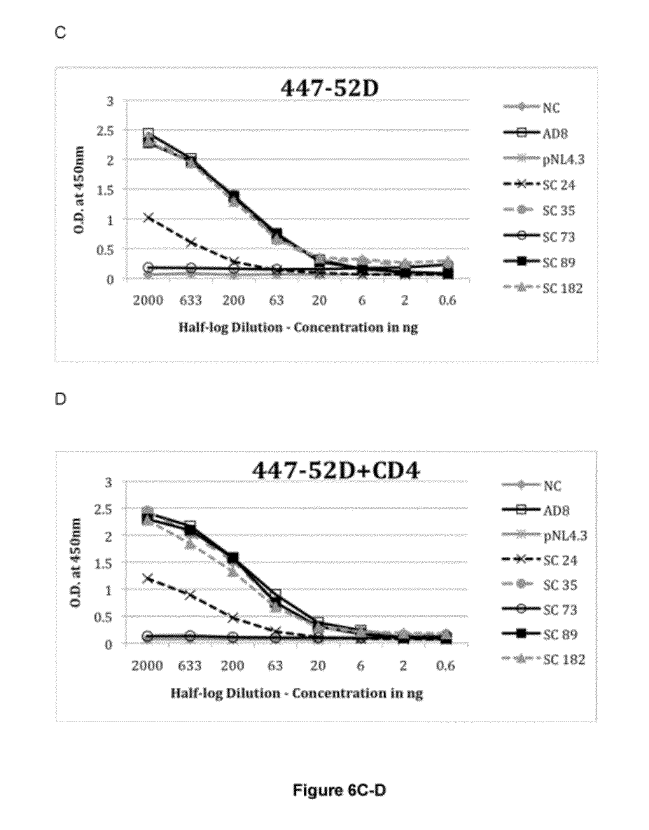

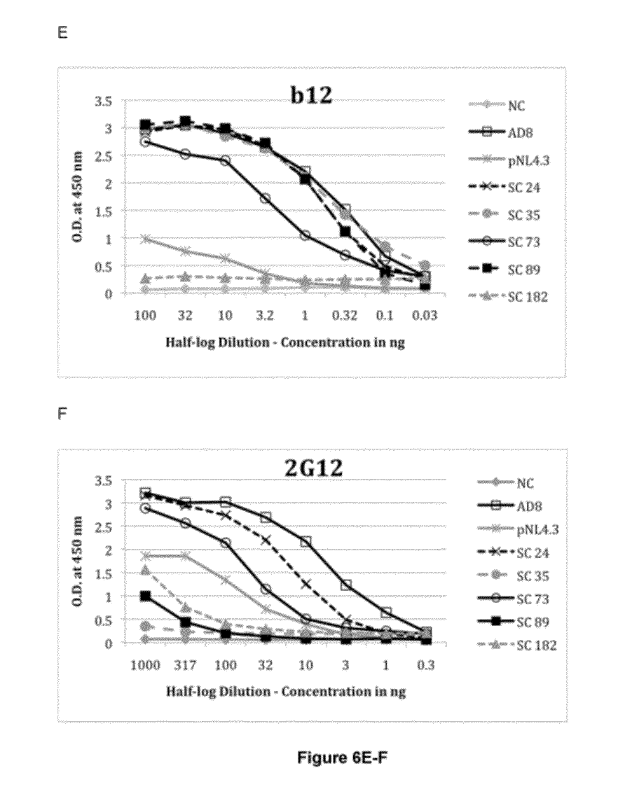

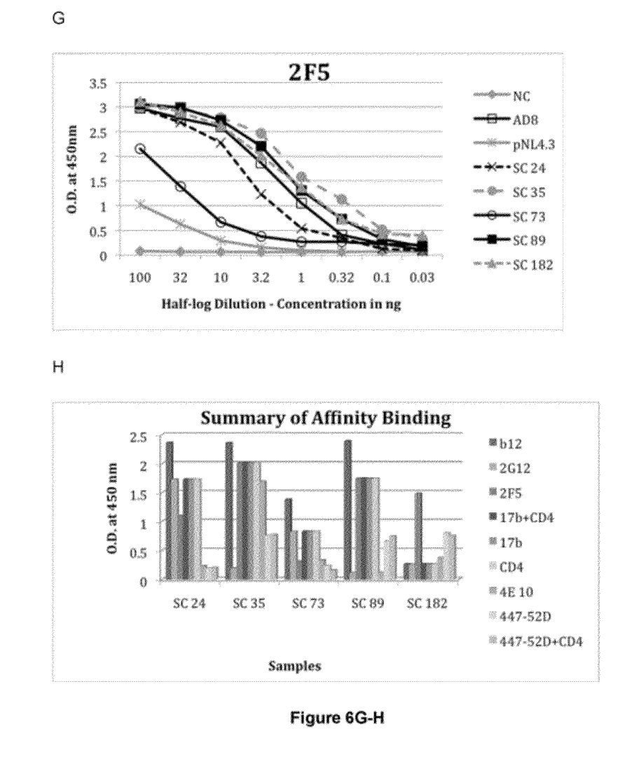

in FIG. 6A, the reference mNab that binds the CD4 induced epitope, 17b, is shown to bind to SC35 Env gp140 without addition of 100 ng soluble CD4 (sCD4-4D) (FIG. 6A). The SC24, SC73 and SC 89 Env gp140 have moderate affinity binding to 17b that is dramtically enhanced with conformational changes to Env gp140 trimers that occur after the addition of sCD4-4D (FIG. 6B). The sCD4-4D that was used was truncated at the transmembrane domain and purified from supernatant from a transduced 293 human cell line using Ni-NTA column, and concentrated using 10,000 NMWL column. These results demonstrate that the all preseroconverson Env gp140, except the non-functional SC182 display the CD4 induced neutralisation epitope that is present on most HIV-1 strains.

In FIG. 6C, the reference mNab that binds the common V3 loop neutralisation epitope, 447-52D binds to SC24, SC35, SC89 and SC 182, but not SC73. There is no change in the binding of this V3 loop binding mNab when 100 ng soluble CD4 (sCD4-4D) was added (FIG. 6D). The Env gp140 have high affinity for this epitope. The results demonstrate that the all preseroconverson Env gp140, except SC73 display the V3 loop neutralisation epitope that is present on most HIV-1 strains.

In FIG. 6E, the reference mNab that binds the common CD4 binding site epitope, b12 binds with high affinity to SC24, SC35, SC89 and SC73, but not the non-functional SC 182. The results demonstrate that the all preseroconverson Env gp140, except non-functional SC182 display the CD4 binding site epitope that is present on most HIV-1 strains.

In FIG. 6F, the reference mNab that binds the common mannose-rich carbohydrate binding site epitope, 2G12 binds with high affinity to SC24 and SC73, low afinity to SC89 but not to SC35. The results demonstrate that the SC24 and SC73 preseroconverson Env gp140, and to a lesser degree SC89 display the mannose-rich carbohydrate dependent 2G12 neutralisation epitope that is present on most HIV-1 strains.

In FIG. 6G, the reference mNab that binds the membrane proximal region epitope, defined by 2F5 binds with high affinity to SC24, SC35, SC89 and SC 182 and to a lesser degree to SC73. The results demonstrate that the all preseroconverson Env gp140, except non-functional SC73 display the membrane proximal region defined by the 2F5 mNab epitope that is preent on most HIV-1 strains.

FIG. 6H is a summary of the binding affinity of each sample for each antibody.

### Example 8

### Vaccination Trial

Using an DNA prime/adjuvanted protein boost vaccination strategy, DNA and protein gp140 formulations of the invention were administered to groups of 8 mice to test for the ability to induce a neutralising antibodies. Control animals received a DNA prime followed by one or more protein boosts of oligomeric gp120 from the AD8 strain of HIV-1 in the form of an uncleaved gp140 protein. In this embodiment the gp120 domains had the N358 variation. Other animals received the transmission strain gp140 oligomers.

Priming with a DNA vaccine followed by boosting with proteins results in the generation of antigen-specific memory T cells by priming followed by amplification of these cells by boosting.

Table 7 summarises the DNA formulations administered to each test mouse. 100 μg of DNA in 100 μL was administered to each mouse via intramuscular injection. The control groups of mice received PBS alone (no DNA—Group 1), empty pN1 vector backbone alone (Group 2) or vector containing wild type gp140 encoding sequence derived from AD8 (Group 3). The test groups received constructs containing gp140 encoding sequence derived from SC24—clone 76 (Group 4), SC35—clone 10 (Group 5), SC73—clone 47 (Group 6) and SC89—clone 51 (Group 7).

TABLE 7
DNA vaccine formulations

| | Group (8 mice/group) | | | | | |
|---|---|---|---|---|---|---|
| | 1 | 2 | 3 | 4 | 5 | 6 |
| DNA | PBS | PN1 | PN1 AD8 | SC24 | SC35 | SC73 |
| | | Empty | gp140 | PN1-gp140 | PN1-gp140 | PN1-gp140 |
| Concentration | | 1 μg/μL | 1 μg/μL | 1 μg/μL | 1 μg/μL | 1 μg/μL |
| Volume/mouse | 100 μl | 100 μl | 100 μl | 100 μl | 100 μl | 100 μl |

Prior to DNA injection, each mouse was anesthetised with the formulations set out in Table 8. The anesthesia was injected into mice by intraperitoneal (IP) route. Of the mice anesthetized, one mouse did not recover.

TABLE 8
Anesthesia formulations

| | | | Dose per | | Product | Mouse |
|---|---|---|---|---|---|---|
| Anesthesia | Quantity | Purpose | animal | | Conc | weight |
| Ketamine | 2.0 mL | Anesthesia | 100 | μg/gm | 100 mg/ml | 20 gms |
| | | | body weight | | (Ilium) | |
| Xylazine | 2.0 mL | Muscle | 10 | μg/gm | 20 mg/ml | |
| | | Relaxation | body weight | | | |
| Saline | 16.0 mL | Dilution | | | | |
| Total | 20.0 mL | | 200 | μL for | | |

each mouse

The protein boost formulations are summarised in Table 9. Freund's incomplete adjuvant (FIA) was used (an antigen solution lacking mycobacterial antigens emulsified in mineral oil used as an immunopotentiator of the immune system) as a control in Group 1 (protein and PBS) and in group 2 with non-HIV derived proteins from 293T and HeLa cell proteins (being the cell lines in which the gp140 proteins were generated). The remaining groups received FIA together with oligomeric gp120 from the AD8 strain of HIV-1 in the form of an uncleaved gp140 protein (SEQ ID NO: 37) (Group 3) or gp120 from each transmission strain sample in the form of an uncleaved gp140 protein (Groups 4 to 6). Each mouse was injected with 200 μl of protein formulations—100 μl via IP followed by 100 μl via subcutaneous route.

TABLE 9
Protein boost formulations with Freund's
Incomplete Adjuvant (FIA)

| Group | 1 | 2 | 3 | 4 | 5 | 6 |
|---|---|---|---|---|---|---|
| Mice | 8 | 8 | 8 | 8 | 8 | 8 |
| Protein | PBS + | 293T + | AD8 | SC24 | SC35 | SC73 |
| Boost | FIA | HeLa | gp140 | gp140 | gp140 | gp140 |
| | | Proteins + | | | | |
| | | FIA | | | | |
| Conc per | PBS 50% | Same as | 10 μg | 10 μg | 10 μg | 10 μg |
| mouse | FIA 50% | 702 | | | | |
| | | (lowest | | | | |
| | | conc/ | | | | |
| | | highest | | | | |
| | | volume) | | | | |
| Volume | 100 μl + | 100 μl + | 100 μl + | 100 μl + | 100 μl + | 100 μl + |
| per mouse | 100 μl | 100 μl | 100 μl | 100 μl | 100 μl | 100 μl |
| Protein + | | | | | | |
| FIA | | | | | | |
| Conc per | | | 380 ng | 330 ng | 250 ng | 200 ng |
| μl | | | | | | |
| Protein | 100 μl | 77 μl | 26.3 μl | 30.3 μl | 40 μl | 50 μl |
| per | | 23 μl | 73.7 μl | 69.7 μl | 60 μl | 50 μl |
| mouse | | | | | | |
| (10 μg) | | | | | | |
| PBS | | | | | | |

The following double DNA prime triple boost regime was followed:

Week 0 Pre vaccination tail bleed (to establish base line)
Week 1 DNA prime
Week 5 DNA prime
Week 7 Tail bleed to check antibody titre
Week 9 Protein boost
Week 11 Tail bleed to check antibody titre
Week 13 Protein boost
Week 15 Tail bleed to check antibody titre
Week 17 Protein boost
Week 19 Tail bleed to check antibody titre
Week 21 Final bleed

Bleeding Technique: All mice were pre bled by tail vein bleed. The mice were placed in a warm box for 10 minutes, prior to the tail vein being nicked with a clean scalpel. Around 100 μL of blood was collected from each test mouse, and following clotting (at room temp), spun at 1500 g for 15 min and the serum stored for testing.

Final bleed: The mice were humanely terminated (via CO2 chamber) and were heart bled. Around 800 μL of blood was collected from each mouse and following clotting, spun at 1500 g for 15 min and the serum stored.

The antibody titers in the serum samples were determined by ELISA assay and then the neutralizing ability of the sera was tested. This assay detects the presence of virus-neutralising antibodies to HIV-1 envelope protein. EGFP reporter pseudovirus particles expressing HIV-1 Env derived from strains of choice were used to infect target cells in an Env dependent manner. The reporter pseudovirus particles are incubated for 1 hour before the addition of the target cells (Cf2th-CD4/CCR5/CXCR4) at 2×10⁴/well in a 96-well plate. After a 2-hour spinoculation at 1200×g at room temperature, residual pseudovirus and antibody was removed and fresh media added to the cells. Two days later the target cells were analysed for EGFP expression by FACS.

In the presence of sera raised against HIV-1 Env, the degree of reduction in the level of infection was determined by measuring the reduction in the percent EGFP positive cells. The neutralisation percentage represents a ratio between infection levels observed in mice sera before vaccination (pre-bleed) and mice sera 2 weeks post protein boost 3 vaccination.

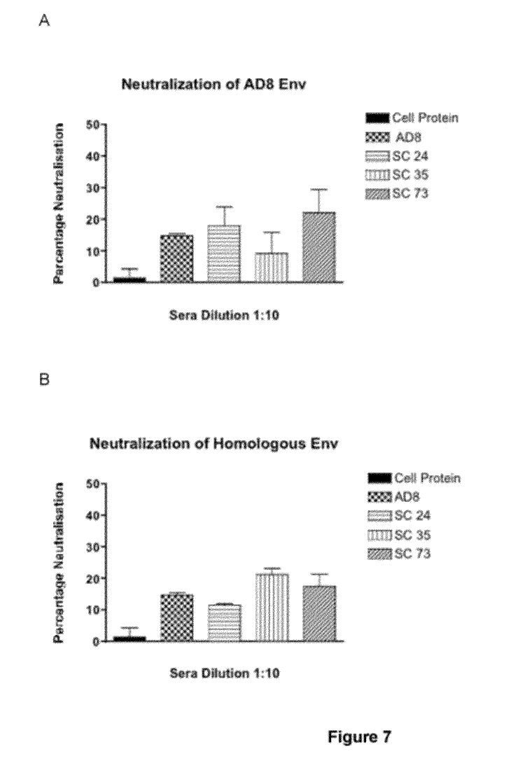

The results of the neutralization assay are presented in FIG. 7A-B. Data were shown for a serum dilution of 1:10. The sera was pooled form 6-8 mice for each group and data represents an average of duplicate measurements. Env gp140 proteins raised a weak neutralising antibody response against the immunogens in the mice system. However, neutralising activity was induced in sera of mice vaccinated with the Env gp140 immunogens and this activity was significant higher then for the control group vaccinated with HeLa and/or 293T cell protein only.

1. A polypeptide comprising an HIV-1 envelope protein from a transmission strain of HIV-1 or a fragment of said protein, the polypeptide comprising neutralization sensitive epitopes that are accessible.

2. A polypeptide according to claim 1, wherein the envelope protein from the transmission strain has an altered glycosylation profile compared to a glycosylation profile of an envelope protein from post-seroconversion strains of HIV-1.

3. A polypeptide according to claim 1, wherein the envelope protein from the transmission strain has one or more altered variable loop sequences compared to variable loop sequences of an envelope protein from post-seroconversion strains of HIV-1.

4. A polypeptide according to claim 1, wherein the envelope protein from the transmission strain has both an altered glycosylation profile and one or more altered variable loop sequences compared to a glycosylation profile and variable loop sequences of an envelope protein from post-seroconversion strains of HIV-1.

5. A polypeptide according to claim 2, wherein the altered glycosylation profile is as a result of deletion, modification, or mutation of one or more glycosylation sites within the HIV-1 envelope polypeptide.

6. A polypeptide according to claim 2, wherein one or more altered glycosylation sites are in a C4 domain and/or one or more variable V1-V5 domains of the HIV-1 envelope polypeptide.

7. A polypeptide according to claim 6, wherein the one or more glycosylation sites are N197, N358 and N386 according to the numbering of SEQ ID NO: 36.

8. A polypeptide according to claim 3, wherein the one or more altered variable loop sequences are compared to variable loop sequences of an envelope protein from post-seroconversion strains of HIV-1 having the same function or having at least 95% sequence identity.

9. A polypeptide according to claim 3 wherein the variable loop is V2.

10. An HIV-1 neutralizing antibody for binding neutralization sensitive epitopes, wherein the neutralization sensitive epitopes are accessible in a transmission strain of HIV-1.

11. An HIV-1 neutralizing antibody according to claim 10, wherein the neutralization epitopes are located in a C2 domain, C4 domain, V3 domain or V4 domain of the HIV envelope polypeptide.

12. An oligomeric polypeptide comprising the polypeptide according to claim 1, wherein the oligomeric polypeptide generates neutralizing antibodies to transmission strains of HIV-1.

13. A method of reducing the risk of HIV infection in a seronegative individual, comprising administering to the individual an HIV-1 envelope polypeptide or polypeptide fragment thereof, wherein one or more glycosylation sites in gp120, gp41, or both are deleted or mutated.

14. A method of reducing the risk of HIV infection in a seronegative individual comprising administering to the individual an HIV-1 polypeptide according to claim 1.

15. A composition for use in raising an immune response comprising an HIV-1 envelope polypeptide or polypeptide fragment, wherein one or more glycosylation sites in gp120, gp41, or both are deleted or mutated.

16. A composition for use in raising an immune response comprising an HIV-1 polypeptide according to claim 1.

17. A composition for use in raising an immune response comprising the neutralizing antibody of claim 10.

18. A composition according to claim 15, further comprising a pharmaceutically carrier, excipient or diluent.

19. A method of treating an HIV-1 seropositive individual to reduce symptoms of HIV-1 infection comprising administering to the individual an HIV-1 polypeptide according to claim 1.

20. A method of treating an HIV-1 seropositive individual to reduce symptoms of HIV-1 infection comprising administering to the individual the neutralizing antibody of claim 10.

21. A method of reducing the risk of HIV infection in a seronegative individual comprising administering to the individual an oligomeric polypeptide according to claim 12.

22. A composition for use in raising an immune response comprising an oligomeric polypeptide according to claim 12.

23. A composition for use in raising an immune response comprising the neutralizing antibody of claim 11.

24. A composition according to claim 16, further comprising a pharmaceutically carrier, excipient or diluent.

25. A composition according to claim 17, further comprising a pharmaceutically carrier, excipient or diluent.

26. A method of treating HIV-1 seropositive individuals to reduce symptoms of HIV-1 infection comprising administering to the individual an oligomeric polypeptide according to claim 12.

27. A method of reducing the risk of HIV infection in a seronegative individual comprising administering to the individual an oligomeric polypeptide according to claim 11.


SEQUENCE LISTING (41 sequences)

SEQ ID NO: 1 - DNA, 2565 bp, Human immunodeficiency virus
atgagagtgaaggagaaatatcagcacttgtggagatgggggtggagatggggcaccatgctccttgggatgttgatgatctgtagtgctacagaaaaattgtgggtcacagtctattatggggtacctgtgtggaaggatgctaacaccactctattttgtgcatcaaatgctaaagcatatgatacagaggcacataatgtttgggccacacacgcctgtgtacccacagaccccaacccacaagaagtagtattgatcaatgtgacagaaaattttaacatgtggaaaaataacatggtagaacaaatgcatgaggatataatcagtttatgggatcaaagcctaaagccatgtgtaaaattaaccccactctgtgttactttaaattgcagtgatgtaaatggcactagtagtaataccactagagagataagaaatgagacaataatggaaaaaggagaagtgaaaaactgctctttcaatatcaccacaagcataagagatagggtgcagaaacaatatgcaacattttatagacttgatgtagtgcagatagatgataatgataatactagttataggatgacaagttgtaacgcctcagtcattacacaggcctgtccaaagatatcctttgagccaattcccatacatttttgtgccccggctggttttgcgattctaaagtgtacagaaaaggggttcaatggaacaggaaaatgtaaaaatgtcagcacagtacaatgtacacatggaattaggccagtagtatcaactcaattgctgttaaatggcagtctagcagaagaaggggtagtaattcgatctcaaaatttaacagacaatactaaaaccataatagtacagctggaaaaagctataaaaattaattgtacaagacccaacaacaatacaagaaaaagtatgagtctaggtccagggaaagtattttatacaacaggacaaataataggagatataagaaaagcacattgtaaccttactgcaggagattggtatagcgctttaaaagagatagccataaaattaagaggacaatttaacaataaaacaatagtctttaagaactcctcaggaggggactcagaaattgtaacccacagttttaattgtggaggggaatttttctactgtgactcaacaaaactgtttaattttacttggaatggtactgaaacaaataacactggaggaagtgataaaatcatactcccatgcagaataaaacaaattataaacatgtggcagaaagtaggacaagcaatgtatgcccctcccatccaaggaaaaatttactgttcatcaaatattacagggctactattaacaagagatggtggcatcaacaatgaaactaacaataccgagaccttcagacctggaggaggaaatatgaaggataattggagaagtgaattatataaatataaagtagtacaaattgaaccattaggagtagcacccaccaaggcaaaaagaagagtggtgcggagagaaaaaagagcggtgggcatgggagctttgttccttgggttcttgggagcagcaggaagcactatgggcgcagcgtcaataacgctgacggtacaggccagacaattattgtctggtatagtgcaacagcagaacaatctgctgagggctgttgaggcgcaacagcatatgttgcaactcacagtctggggcatcaagcagctccaggcgagagtcctggctgtggaaagatacctaagggatcaacagctcctggggctctggggttgctctggaaagctcatttgcaccactactgtgccttggaatactagttggagtaataaatctctgaatgagatttgggataacatgacctggaaggaatgggacagagaaattaacaagtacacaaatgtaatatactccttaattgaagaatcgcagaaccagcaagaaaagaatgaacaagaattattggcattagataagtgggcaagtttgtggaattggtttgacataacaaaatggctgtggtatataaagatattcataatgatagtaggaggcttgataggtttaagaatagtttttagtgtactttctatagtgaatagagttaggcagggatactcacctttatcgtttcagacccacctcccagctcagaggggacccgacaggcccgaaggaatcgaagaagaaggtggagagagagacagggacagatccggaagattagtggatggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataa

SEQ ID NO: 2 - protein, 854 aa, Human immunodeficiency virus
MRVKEKYQHLWRWGWRWGTMLLGMLMICSATEKLWVTVYYGVPVWKDANTTLFCASNAKAYDTEAHNVWATHACVPTDPNPQEVVLINVTENFNMWKNNMVEQMHEDIISLWDQSLKPCVKLTPLCVTLNCSDVNGTSSNTTREIRNETIMEKGEVKNCSFNITTSIRDRVQKQYATFYRLDVVQIDDNDNTSYRMTSCNASVITQACPKISFEPIPIHFCAPAGFAILKCTEKGFNGTGKCKNVSTVQCTHGIRPVVSTQLLLNGSLAEEGVVIRSQNLTDNTKTIIVQLEKAIKINCTRPNNNTRKSMSLGPGKVFYTTGQIIGDIRKAHCNLTAGDWYSALKEIAIKLRGQFNNKTIVFKNSSGGDSEIVTHSFNCGGEFFYCDSTKLFNFTWNGTETNNTGGSDKIILPCRIKQIINMWQKVGQAMYAPPIQGKIYCSSNITGLLLTRDGGINNETNNTETFRPGGGNMKDNWRSELYKYKVVQIEPLGVAPTKAKRRVVRREKRAVGMGALFLGFLGAAGSTMGAASITLTVQARQLLSGIVQQQNNLLRAVEAQQHMLQLTVWGIKQLQARVLAVERYLRDQQLLGLWGCSGKLICTTTVPWNTSWSNKSLNEIWDNMTWKEWDREINKYTNVIYSLIEESQNQQEKNEQELLALDKWASLWNWFDITKWLWYIKIFIMIVGGLIGLRIVFSVLSIVNRVRQGYSPLSFQTHLPAQRGPDRPEGIEEEGGERDRDRSGRLVDGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 3 - DNA, 2565 bp, Human immunodeficiency virus; identical to SEQ ID NO: 1

SEQ ID NO: 4 - protein, 854 aa, Human immunodeficiency virus; identical to SEQ ID NO: 2

SEQ ID NO: 5 - DNA, 2577 bp, Human immunodeficiency virus
atgagagtgaaggagaaatatcagcacttgtggagatgggggtggagatggggcaccatgctccttgggatgttgatgatctgtagtgctacagaaaaattgtgggtcacagtctattatggggtacctgtgtggaaagaagcaactaccactctattttgtgcatcagatgctaaagcatatgatacagaggtacataatgtttgggccacacatgcctgtgtacccacagaccccaacccacaagaagtagtattgggaaatgtaacagaatattttaacatgtggaaaaatagtatggtggaacagatgcatgaggatataatcagtctatgggatgaaagcttaaagccatgcgtaaaattaaccccactctgtgtttctttaaattgcactgattggaagaatagtactaatacccataataacagttgtggaagtggttcaaacaactgctctttggaaatggagaaaggagaaataaaaaactgctctttcaatatcacctcacgcatgagagataggacgcaggaaaaatatgcacttttttataaacttgatgtagtacaaatagatggtgataagggtaataccagcagctataggttgatacattgtaatacctcagtcattacacaggcctgtccaaaggtatcctttgagccaattcccatacattattgtaccccggctggttttgcgattctaaagtgtaacgataagaggttcaagggaaaaggacaatgtacaaatgtcagcacagtacagtgtacacatggaattaggccagtggtgtcaactcaattgctgttaaatggcagcctagcagaagaggagataataattaggtctgacaatatcacggacaatgctaaaaccataatagtacagctgaatgaatctgtagcaattacatgtgaaagaccaggcaacaatacaagaagaagtatacctataggaccagggagagtattttatacaggagaaataacaggagatataagaaaagcacattgcaatattactaaaaaaggctgggagagcactttaaaaaagatagctagcaagttaaaagaacaatttaataagacaatagtctttaatcaatcctcaggaggggacccagaaattgtaatgcacagttttaattgtagaggggaatttttctactgtgattcagcacagctgtttaatagtacttggccgtctaatagtactggggaaacaaatgacactcaaggaatcaatatcacactcccatgtagaataaaacaaattataaacatgtggcagggagtaggaaaagcaatgtatgcccctcccatcagaggacgaattacatgtacatcaaatattacagggctgctgttaacaagggatggtggtcataacacaagcaaggagaccttcagacctggaggaggaaatatgagggacaattggagaagtgaactatataaatataaagtagtaaaaattgaaccattaggaatagcacccaccaaggcaaagagaagagtggtgcagagagaaaaaagaggagtaacaataggagctatgttccttgggttcttgggagcagcaggaagcactatgggcgcagcgtcaatggcgctgacggcacaggccagacaattattgtctggtatagtgcaacagcagaacaatctgctgagggttattgaggtgcaacagcatatgttgcaactcacagtctggggcatcaagcagctccaggcaagagtcctggctgtggaaagatacctaagggatcaacagctcctagggatttggggttgctctggaaaactcatttgcaccactgctgtaccttggaatgctagttggagcaataaatctcaggaggatatttgggaaaacatgacctggatgcagtgggaaaaagaaattgacaattacacagggttaatctacaccttacttgaaaaatcgcagaaccagcaagaaaagaatgaacaagaattattggaattggataagtgggcaggtttgtggaattggtttgacataacacaatggctgtggtatataaaaatattcataatgatagtaggaggcttgataggtttaagaatagtttttgctgtattttctatagtaaatagagttaggcagggatactcaccattgtcatttcagacccacctcccagttccgaagggacccgaacggccagaaggaaccgaagaaggaggtggagagagagacagagacagatccgtacgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataa

SEQ ID NO: 6 - protein, 858 aa, Human immunodeficiency virus
MRVKEKYQHLWRWGWRWGTMLLGMLMICSATEKLWVTVYYGVPVWKEATTTLFCASDAKAYDTEVHNVWATHACVPTDPNPQEVVLGNVTEYFNMWKNSMVEQMHEDIISLWDESLKPCVKLTPLCVSLNCTDWKNSTNTHNNSCGSGSNNCSLEMEKGEIKNCSFNITSRMRDRTQEKYALFYKLDVVQIDGDKGNTSSYRLIHCNTSVITQACPKVSFEPIPIHYCTPAGFAILKCNDKRFKGKGQCTNVSTVQCTHGIRPVVSTQLLLNGSLAEEEIIIRSDNITDNAKTIIVQLNESVAITCERPGNNTRRSIPIGPGRVFYTGEITGDIRKAHCNITKKGWESTLKKIASKLKEQFNKTIVFNQSSGGDPEIVMHSFNCRGEFFYCDSAQLFNSTWPSNSTGETNDTQGINITLPCRIKQIINMWQGVGKAMYAPPIRGRITCTSNITGLLLTRDGGHNTSKETFRPGGGNMRDNWRSELYKYKVVKIEPLGIAPTKAKRRVVQREKRGVTIGAMFLGFLGAAGSTMGAASMALTAQARQLLSGIVQQQNNLLRVIEVQQHMLQLTVWGIKQLQARVLAVERYLRDQQLLGIWGCSGKLICTTAVPWNASWSNKSQEDIWENMTWMQWEKEIDNYTGLIYTLLEKSQNQQEKNEQELLELDKWAGLWNWFDITQWLWYIKIFIMIVGGLIGLRIVFAVFSIVNRVRQGYSPLSFQTHLPVPKGPERPEGTEEGGGERDRDRSVRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 7 - DNA, 2577 bp, Human immunodeficiency virus
atgagagtgaaggagaaatatcagcacttgtggagatgggggtggagatggggcaccatgctccttgggatgttgatgatctgtagtgctacagaaaaattgtgggtcacagtctattatggggtacctgtgtggaaagaagcaactaccactctattttgtgcatcagatgctaaagcatatgatacagaggtacataatgtttgggccacacatgcctgtgtacccacagaccccaacccacaagaagtagtattgggaaatgtaacagaatattttaacatgtggaaaaatagtatggtggaacagatgcatgaggatataatcagtctatgggatgaaagcttaaagccatgtgtaaaattaaccccactctgtgtttctttaaattgcactgattggaagaatagtactaatacccataataacagttgtggaagtggttcaaacaactgctctttggaaatggagaaaggagaaataaaaaactgctctttcaatatcacctcacgcatgagagataggacgcaggaaaaatatgcacttttttataaacttgatgtagtacaaatagatggtgataagggtaataccagcagctataggttgatacattgtaatacctcagtcattacacaggcctgtccaaaggtatcctttgagccaattcccatacattattgtaccccggctggttttgcgattctaaagtgtaacgataagaggttcaagggaaaaggacaatgtacaaatgtcagcacagtacagtgtacacatggaattaggccagtggtgtcaactcaattgctgttaaatggcagcctagcagaagaggggataataattaggtctgacaatatcacggacaatgctaaaaccataatagtacagctgaatgaatctgtagcaattacatgtgaaagaccaggcaacaatacaagaagaagtatacctataggaccagggagagtattttatacaggagaaataacaggagatataagaaaagcacattgcaatattactaaaaaaggctgggagagcactttaaaaaagatagctagcaagttaaaagaacaatttaataagacaatagtctttaatcaatcctcaggaggggacccagaaattgtaatgcacagttttaattgtagaggggaatttttctactgtgattcagcacagctgtttaatagtacttggccgtctaatagtactggggaaacaaatgacactcaaggaatcaatatcacactcccatgtagaataaaacaaattataaacatgtggcagggagtaggaaaagcaatgtatgcccctcccatcagaggacgaattacatgtacatcaaatattacagggctgctgttaacaagggatggtggtcataacacaagcaaggagaccttcagacctggaggaggaaatatgagggacaattggagaagtgaactatataaatataaagtagtaaaaattgaaccattaggaatagcacccaccaaggcaaagagaagagtggtgcagagagaaaaaagaggagtaacaataggagctatgttccttgggttcttgggagcagcaggaagcactatgggcgcagcgtcaatggcgctgacggcacaggccagacaattattgtctggtatagtgcaacagcagagcaatctgctgagggctattgaggcgcaacagcatatgttgcaactcacagtctggggcatcaagcagctccaggcaagagtcctggctgtggaaagatacctaagggatcaacagctcctagggatttggggttgctctggaaaactcatttgcaccactgctgtgccttggaatgctagttggagcaataaatctcaggaggatatttgggaaaacatgacctggatgcagtgggaaaaagaaattgacaattacacagggttaatctacaccttacttgaaaaatcgcagaaccagcaagaaaagaatgaacaagaattattggaattggataagtgggcaggtttgtggaattggtttgacataacacaatggctgtggtatataaaaatattcataatgatagtaggaggcttgataggtttaagaatagtttttgctgtattttctatagtaaatagagttaggcagggatactcaccattgtcatttcagacccacctcccagtcccgaggggacccgaacggccagaaggaaccgaagaaggaggtggagagagagacagagacagatccgtacgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataa

SEQ ID NO: 8 - protein, 858 aa, Human immunodeficiency virus
MRVKEKYQHLWRWGWRWGTMLLGMLMICSATEKLWVTVYYGVPVWKEATTTLFCASDAKAYDTEVHNVWATHACVPTDPNPQEVVLGNVTEYFNMWKNSMVEQMHEDIISLWDESLKPCVKLTPLCVSLNCTDWKNSTNTHNNSCGSGSNNCSLEMEKGEIKNCSFNITSRMRDRTQEKYALFYKLDVVQIDGDKGNTSSYRLIHCNTSVITQACPKVSFEPIPIHYCTPAGFAILKCNDKRFKGKGQCTNVSTVQCTHGIRPVVSTQLLLNGSLAEEGIIIRSDNITDNAKTIIVQLNESVAITCERPGNNTRRSIPIGPGRVFYTGEITGDIRKAHCNITKKGWESTLKKIASKLKEQFNKTIVFNQSSGGDPEIVMHSFNCRGEFFYCDSAQLFNSTWPSNSTGETNDTQGINITLPCRIKQIINMWQGVGKAMYAPPIRGRITCTSNITGLLLTRDGGHNTSKETFRPGGGNMRDNWRSELYKYKVVKIEPLGIAPTKAKRRVVQREKRGVTIGAMFLGFLGAAGSTMGAASMALTAQARQLLSGIVQQQSNLLRAIEAQQHMLQLTVWGIKQLQARVLAVERYLRDQQLLGIWGCSGKLICTTAVPWNASWSNKSQEDIWENMTWMQWEKEIDNYTGLIYTLLEKSQNQQEKNEQELLELDKWAGLWNWFDITQWLWYIKIFIMIVGGLIGLRIVFAVFSIVNRVRQGYSPLSFQTHLPVPRGPERPEGTEEGGGERDRDRSVRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 9 - DNA, 2565 bp, Human immunodeficiency virus; identical to SEQ ID NO: 1

SEQ ID NO: 10 - protein, 854 aa, Human immunodeficiency virus; identical to SEQ ID NO: 2

SEQ ID NO: 11 - DNA, 2565 bp, Human immunodeficiency virus; identical to SEQ ID NO: 1

SEQ ID NO: 12 - protein, 854 aa, Human immunodeficiency virus; identical to SEQ ID NO: 2

SEQ ID NO: 13 - DNA, 2565 bp, Human immunodeficiency virus; identical to SEQ ID NO: 1

SEQ ID NO: 14 - protein, 854 aa, Human immunodeficiency virus; identical to SEQ ID NO: 2

SEQ ID NO: 15 - DNA, 2565 bp, Human immunodeficiency virus
atgagagtgaaggagaaatatcagcacttgtggagatgggggtggagatggggcaccatgctccttgggatgttgatgatctgtagtgctacagaaaaattgtgggtcacagtctattatggggtacctgtgtggagagaagcaaccaccactctattttgtgcatcagatgctaaagcctatgatacagaggtacataatgtttgggccacacatgcctgtgtacccacagaccccaacccacaagaagtagtattgggaaatgtgacagaaaattttaacatgtggaaaaataacatggtagatcagatgcatgaggatataatcagtttatgggatgaaagcctaaagccatgtgtaaaattaaccccactctgtgttactttaaattgcactaatttgaatatcactaagaatactactaatcccactagtagcagctggggaatgatggagaaaggagaaataaaaaattgctctttctatatcaccacaagcataagaaataaggtaaagaaagaatatgcactttttaatagacttgatgtagtaccaatagaaaatactaataatactaagtaccggttaataagttgtaacacctcagtcattacacaggcctgtccaaaggtatcctttcagccaattcccatacattattgtgtcccggctgggtttgcgatgctaaagtgtaacaataagacattcaatggatcaggaccatgcacaaatgtcagcacagtacaatgtacacatggaattaggccagtggtgtcaactcaactgctgttaaatggcagtctagcagaagaagacatagtaattagatctgaaaatttcacagacaatgctaaaaccataatagtacagctaaatgaatctgtagtaattaattgtacaagacccaacaacaatacaagaagaaggttatctataggaccagggagagcattttatgcaagaagaaacataataggagatataagacaagcacattgtaacattagtagagcaaaatggaataacactttacaacagatagttataaaattaagagaaaaatttaggaataaaacaatagcctttaatcaatcctcaggaggggacccagaaattgtaatgcacagttttaattgtggaggggaatttttctactgtaatacagcacaactgtttaatagtacttggaatgttactggagggacaaatggcactgaaggaaatgacataatcacactccaatgcagaataaaacaaattataaatatgtggcagaaagtaggaaaagcaatgtatgcccctcccatcacaggacaaattagatgttcatcaaatattacagggctgctactaacaagagatggaggtaatagtactgagactgagactgagatcttcagacctggaggaggagatatgagggacaattggagaagtgaattatataaatataaagtagtaagaattgaaccaataggagtagcacccaccagggcaaagagaagaacagtgcaaagagaaaaaagagcagtgggaataggagctgtgttccttgggttcttgggagcagcaggaagcactatgggcgcagcgtcagtgacgctgacggtacaggccaggctattattgtctggtatagtgcagcagcagaacaatctgctgagggctattgaggcgcaacagcatatgttgcaactcacagtctggggcatcaagcagctccaggcaagagtcctggctctggaaagatacctaagggatcaacagctcatgggaatttggggttgctctggaaaactcatttgcaccacttctgtgccttggaatgttagttggagtaataaatctgtggatgatatttggaataacatgacctggatggagtgggaaagagaaattgacaattacacagactatatatatgacttacttgaaaaatcgcaaacccaacaagaaaagaatgaaaaagaattattggaattggataaatgggcaagtttgtggaattggtttgacataacaaactggctgtggtatataagattattcataatgatagtaggaggcttgataggtttaagaatagtttttgctgtactttctatagtaaatagagttaggcagggatattcaccattatcgtttcagaccctcctcccagcctcgaggggacccgacaggcccgaaggaacagaagaagaaggtggagagagagacagagacagatccggtccattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataa

SEQ ID NO: 16 - protein, 854 aa, Human immunodeficiency virus
MRVKEKYQHLWRWGWRWGTMLLGMLMICSATEKLWVTVYYGVPVWREATTTLFCASDAKAYDTEVHNVWATHACVPTDPNPQEVVLGNVTENFNMWKNNMVDQMHEDIISLWDESLKPCVKLTPLCVTLNCTNLNITKNTTNPTSSSWGMMEKGEIKNCSFYITTSIRNKVKKEYALFNRLDVVPIENTNNTKYRLISCNTSVITQACPKVSFQPIPIHYCVPAGFAMLKCNNKTFNGSGPCTNVSTVQCTHGIRPVVSTQLLLNGSLAEEDIVIRSENFTDNAKTIIVQLNESVVINCTRPNNNTRRRLSIGPGRAFYARRNIIGDIRQAHCNISRAKWNNTLQQIVIKLREKFRNKTIAFNQSSGGDPEIVMHSFNCGGEFFYCNTAQLFNSTWNVTGGTNGTEGNDIITLQCRIKQIINMWQKVGKAMYAPPITGQIRCSSNITGLLLTRDGGNSTETETEIFRPGGGDMRDNWRSELYKYKVVRIEPIGVAPTRAKRRTVQREKRAVGIGAVFLGFLGAAGSTMGAASVTLTVQARLLLSGIVQQQNNLLRAIEAQQHMLQLTVWGIKQLQARVLALERYLRDQQLMGIWGCSGKLICTTSVPWNVSWSNKSVDDIWNNMTWMEWEREIDNYTDYIYDLLEKSQTQQEKNEKELLELDKWASLWNWFDITNWLWYIRLFIMIVGGLIGLRIVFAVLSIVNRVRQGYSPLSFQTLLPASRGPDRPEGTEEEGGERDRDRSGPLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 17 - DNA, 2448 bp, Human immunodeficiency virus
atgagagtgaaggagaaatatcagcacttgtggagatgggggtggagatggggcaccatgctccttgggatgttgatgatctgtagtgctacagaaaaattgtgggtcacagtctattatggggtacctgtgtggaaagaagcaaataccactctattttgtgcatcagatgctaaagcatatgattcagaggtacataatgtttgggctacacatgcctgtgtacccacagaccccaatccacaagaagtattattgggaaatgtgacagaaaattttaatatgtggaaaaataacatggtagaacagatgcatgaagatataatcagtttatgggatcaaagcctaaagccatgtgtaaaattaaccccactctgtgttactttaaattgcactgatttgattagcactgagtcggagaatattactgatgccattaatgagaaaggagaaataaaaaactgctcgttcaatatcaccacaagagtaggaaataagttgaagggagaatatgcatttttttataaccttgatgtagtatcaataaatgatgacaataacaataatacttataggttgataaattgtaatacctcagtcattacacaggcctgtccaaaggtatcctttgagccaattcctatacattattgtgccccggctggttttgcgattctaaagtgtaacgataagaaattcaatggaaaaggacaatgtacaaatgtcagcacagtacaatgtacacatggaattaggccagtagtgtcaactcaactgctgttaaatggcagtctagcagaagaagaagtagtaattagatctgataatatcacgaataatgttaaaatcataatagtacagctgaatgaatctgtagaaatcaagtgtgcaagacccaacaacaatacaagaaaaagtatacctgtaggaccagggcgagcaatttatgcaacaggagacataataggagatataagacaagcacattgtaacattagtagagcacaatggaataacactttaaaacagatagcaagaaaactaagggaacaattcaacaataaaacaatagtgtttaagccatcctcaggaggggacccagaaattgtaatgcacagttttaattgtagaggggaatttttctactgtaattcaacacaactgtttaatagtacttggaatgaaactgctgggaatgatactatagggtcagataacacaactatcacactcccatgcagaataaaacaaattataaacaggtggcaggaagtaggaaaagcaatgtatgcccctcccatcagtggacaaattacatgctcatcaaatattacagggctactattaacaagggatggtggtaaagataataacacaaatgacactactgagatcttcagacctgcagggggaaatatgaaggacaattggagaagtgagttatataaatataaagtagtaaaaattgaaccattaggagtagcacccaccaaggcaaagagaagagtggtgcagagagaaaaaagagcagtgggaacattaggagctatgttccttgggttcttgggagcagcaggaagcactatgggcgcagcgtcaatgacgctgacggtacaggccagactattattgtctggtatagtgcaacagcagaacaatttgctgaaagctattgaggcgcaacagcatctgttgcaactcacagtctggggcatcaagcagctccaggcaagagtcctggctgtggaaagatacctaaaggatcaacagctcctagggatttggggttgctctggaaagctcatttgcaccactgctgtgccttggaatactagttggagtaatagatctctggatatgatttggaataacatgacctggatgcagtgggaaaaagaagttagcaattacacaagtttaatatacgccttaattgaagaatcgcaaaaccaacaagaaaagaatgaacaagaattattggaattagataaatgggcaagtttgtggacttggtttgacataacagactggctgtggtatataaaaatattcataatggtagtaggaggcttgataggtttaagaataatttttgctgtgctttctatagtgaatagagttaggcagggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataa

SEQ ID NO: 18 - protein, 815 aa, Human immunodeficiency virus
MRVKEKYQHLWRWGWRWGTMLLGMLMICSATEKLWVTVYYGVPVWKEANTTLFCASDAKAYDSEVHNVWATHACVPTDPNPQEVLLGNVTENFNMWKNNMVEQMHEDIISLWDQSLKPCVKLTPLCVTLNCTDLISTESENITDAINEKGEIKNCSFNITTRVGNKLKGEYAFFYNLDVVSINDDNNNNTYRLINCNTSVITQACPKVSFEPIPIHYCAPAGFAILKCNDKKFNGKGQCTNVSTVQCTHGIRPVVSTQLLLNGSLAEEEVVIRSDNITNNVKIIIVQLNESVEIKCARPNNNTRKSIPVGPGRAIYATGDIIGDIRQAHCNISRAQWNNTLKQIARKLREQFNNKTIVFKPSSGGDPEIVMHSFNCRGEFFYCNSTQLFNSTWNETAGNDTIGSDNTTITLPCRIKQIINRWQEVGKAMYAPPISGQITCSSNITGLLLTRDGGKDNNTNDTTEIFRPAGGNMKDNWRSELYKYKVVKIEPLGVAPTKAKRRVVQREKRAVGTLGAMFLGFLGAAGSTMGAASMTLTVQARLLLSGIVQQQNNLLKAIEAQQHLLQLTVWGIKQLQARVLAVERYLKDQQLLGIWGCSGKLICTTAVPWNTSWSNRSLDMIWNNMTWMQWEKEVSNYTSLIYALIEESQNQQEKNEQELLELDKWASLWTWFDITDWLWYIKIFIMVVGGLIGLRIIFAVLSIVNRVRQGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 19 - DNA, 2568 bp, Human immunodeficiency virus
atgagagtgaaggagaaatatcagcacttgtggagatgggggtggagatggggcaccatgctccttgggatgttgatgatctgtagtgctacagaaaaattgtgggtcacagtctattatggggtacctgtgtggaaagaagcaaataccactctattttgtgcatcagatgctaaagcatatgattcagaggtacataatgtttgggctacacatgcctgtgtacccacagaccccaacccacaagaagtattattgggaaatgtgacagaaaattttaatatgtggaaaaataacatggtagaacagatgcatgaagatataatcagtttatgggatcaaagcctgaagccatgtgtaaaattaaccccactctgtgttactttaaattgcactgatttgattagcactgagtcggagaatattactgatgccattaatgagaaaggagaaataaaaaactgctcgttcaatatcaccacaagagtaggaaataagttgaagagagaatatgcatttttttataaccttgatgtcgtatcaataaatgatgacaataacaataatacttataggttgataaattgtaatacctcagtcattacacaggcctgtccaaaggtatcctttgagccaattcctatacattattgtgccccggctggttttgcgattctaaagtgtaacgataagaaattcaatggaaaaggacaatgtacaaatgtcagcacagtacaatgtacacatggaattaggccagtagtgtcaactcaactgctgttaaatggcagtctagcagaagaagaagtagtaattagatctgataatatcacgaataatgttaaaatcataatagtacagctgaatgaatctgtagaaatcaagtgtgcaagacccaacaacaatacaagaaaaagtatacctgtaggaccagggcgagcaatttatgcgacaggagacataataggagatataagacaagcacattgtaacattagtagggcacaatggaataacactttaaaacagatagcaagaaaactaagggaacaattcaacaataaaacaatagtgtttaagccatcctcaggaggggacccagaaattgtaatgcacagttttaattgtagaggggaatttttctactgtaattcaacacaactgtttaatagtacttggaatgaaactgctgggaatgatactatagggtcagataacacaactatcacactcccatgcagaataaaacaaattataaacaggtggcaggaagtaggaaaagcaatgtatgcccctcccatcagtggacaaattacatgctcatcaaatattacagggctactattaacaagggatggtggtaaagataataacacaaatgacactactgagatcttcagacctgcagggggaaatatgagggacaattggagaagtgagttatataaatataaagtagtaaaaattgaaccattaggagtagcacccaccaaggcaaagagaagagtggtgcagagagaaaaaagagcagtgggaacattaggagctatgttccttgggttcttgggagcagcaggaagcactatgggcgcagcgtcaatgacgctgacggtacaggccagactattattgtctggtatagtgcaacagcagaacaatttgctgaaagctattgaggcgcaacagcatctgttgcaactcacagtctggggcatcaagcagctccaggcaagagtcctggctgtggaaagatacctaaaggatcaacagctcctagggatttggggttgctctggaaagctcatttgcaccactgctgtgccttggaatactagttggagtaatagatctctggatatgatttggaataacatgacctggatgcagtgggaaaaagaagttagcaattacacaagtttaatatacaccttaattgaagaatcgcaaaaccaacaagaaaagaatgaacaagaattattggaattagataaatgggcaagtttgtggacttggtttgacataacaaactggctgtggtatataaaaatattcataatggtagtaggaggcttgataggtttaagaataatttttgctgtgctttctatagtgaatagagttaggcagggatactcaccattatcatttcagaccctcctcccagctccgaggggacccgacaggcccggaggaatcgaagaagaaggtggagagagagacagaggcagatccgaacgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataa

SEQ ID NO: 20 - protein, 855 aa, Human immunodeficiency virus
MRVKEKYQHLWRWGWRWGTMLLGMLMICSATEKLWVTVYYGVPVWKEANTTLFCASDAKAYDSEVHNVWATHACVPTDPNPQEVLLGNVTENFNMWKNNMVEQMHEDIISLWDQSLKPCVKLTPLCVTLNCTDLISTESENITDAINEKGEIKNCSFNITTRVGNKLKREYAFFYNLDVVSINDDNNNNTYRLINCNTSVITQACPKVSFEPIPIHYCAPAGFAILKCNDKKFNGKGQCTNVSTVQCTHGIRPVVSTQLLLNGSLAEEEVVIRSDNITNNVKIIIVQLNESVEIKCARPNNNTRKSIPVGPGRAIYATGDIIGDIRQAHCNISRAQWNNTLKQIARKLREQFNNKTIVFKPSSGGDPEIVMHSFNCRGEFFYCNSTQLFNSTWNETAGNDTIGSDNTTITLPCRIKQIINRWQEVGKAMYAPPISGQITCSSNITGLLLTRDGGKDNNTNDTTEIFRPAGGNMRDNWRSELYKYKVVKIEPLGVAPTKAKRRVVQREKRAVGTLGAMFLGFLGAAGSTMGAASMTLTVQARLLLSGIVQQQNNLLKAIEAQQHLLQLTVWGIKQLQARVLAVERYLKDQQLLGIWGCSGKLICTTAVPWNTSWSNRSLDMIWNNMTWMQWEKEVSNYTSLIYTLIEESQNQQEKNEQELLELDKWASLWTWFDITNWLWYIKIFIMVVGGLIGLRIIFAVLSIVNRVRQGYSPLSFQTLLPAPRGPDRPGGIEEEGGERDRGRSERLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 21 - DNA, 2568 bp, Human immunodeficiency virus
atgagagtgaaggagaaatatcagcacttgtggagatgggggtggagatggggcaccatgctccttgggatgttgatgatctgtagtgctacagaaaaattgtgggtcacagtctattatggggtacctgtgtggaaagaagcaaataccactctattttgtgcatcagatgctaaagcatatgattcagaggtacataatgtttgggctacacatgcctgtgtacccacagaccccaacccacaagaagtattattgggaaatgtgacagaaaattttaatatgtggaaaaataacatggtaaaacagatgcatgaagatataatcagtttatgggatcaaagcctaaagccatgtgtaaaattaaccccactctgtgttactttaaattgcactgatttgattagcactgagtcggagaatattactgatgccattaatgagaaaggagaaataaaaaactgctcgttcaatatcaccacaagagtaggaaataagttgaagagagaatatgcatttttttataaccttgatgtagtatcaataaataatgacaataacaataatacttataggttgataaattgtaatacctcagtcattacacaggcctgtccaaaggtatcctttgagccaattcctatacattattgtgccccggctggttttgcgattctaaagtgtaacgataagaaattcaatggaaaaggacaatgtacaaatgtcagcacagtacaatgtacacatggaattaggccagtagtgtcaactcaactgctgttaaatggcagtctagcagaagaagaagtagtaattagatctgataatatcacgaataatgttaaaatcataatagtacagctgaatgaatctgtagaaatcaagtgtgcaagacccaacaacaatacaagaaaaagtatacctgtaggaccagggcgagcaatttatgcaacaggagacataataggagatataagacaagcacattgtaacattagtagagcacaatggaataacactttaaaacagatatcaagaaaactaagggaacaattcaacaataaaacaatagtgtttaagccatcctcaggaggggacccagaaattgtaatgcacagttttaattgtagaggggaatttttctactgtaattcaacacaactgtttaatagtacttggaatgaaactgctgggaatgatactatagggtcagataacacaactatcacactcccatgcagaataaaacaaattataaacaggtggcaggaagtaggaaaagcaatgtatgcccctcccatcagtggacaaattacatgctcatcaaatattacagggctactattaacaagggatggtggtaaagataataacacaaatgacactactgagatcttcagacctgcagagagaaatatgaaggacaattggagaagtgagttatataaatataaagtagtaaaaattgaaccattaggagtagcacccaccaaggcaaagagaagagtggtgcagagagaaaaaagagcagtgggaacattaagagctatgttccttgggttcttgggagcagcgggaagcactatgggcgcagcgtcaatgacgctgacggtacaggccagactattattgtctggtatagtgcaacagcagaacaatttgctgaaagctattgaggcgcaacagcatctgttgcaactcacagtctggggcatcaagcagctccaggcaagagtcctggctgtggaaagatacctaaaggatcaacagctcctagggatttggggttgctctggaaagctcatttgcaccactgctgtgccctggaatactagttggagtaatagatctctggatatgatttggaataacatgacctggatgcagtgggaaaaagaagttagcaattacacaagtttaatatacaccttaattgaagaatcgcaaaaccaacaagaaaagaatgaacaagaattattagaattagataaatgggcaagtttgtggacttggtttgacataacaaactggctgtggtatataaaaatattcataatggtagtaggaggcttgataggtttaagaataatttttgctgtgctttctatagtgaatagagttaggcagggatactcaccattatcatttcagaccctcctcccagctccgaggggacccgacaggcccggaggaatcgaagaagaaggtggagagagagacagaggcagatccgatcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataa

SEQ ID NO: 22 - protein, 855 aa, Human immunodeficiency virus
MRVKEKYQHLWRWGWRWGTMLLGMLMICSATEKLWVTVYYGVPVWKEANTTLFCASDAKAYDSEVHNVWATHACVPTDPNPQEVLLGNVTENFNMWKNNMVKQMHEDIISLWDQSLKPCVKLTPLCVTLNCTDLISTESENITDAINEKGEIKNCSFNITTRVGNKLKREYAFFYNLDVVSINNDNNNNTYRLINCNTSVITQACPKVSFEPIPIHYCAPAGFAILKCNDKKFNGKGQCTNVSTVQCTHGIRPVVSTQLLLNGSLAEEEVVIRSDNITNNVKIIIVQLNESVEIKCARPNNNTRKSIPVGPGRAIYATGDIIGDIRQAHCNISRAQWNNTLKQISRKLREQFNNKTIVFKPSSGGDPEIVMHSFNCRGEFFYCNSTQLFNSTWNETAGNDTIGSDNTTITLPCRIKQIINRWQEVGKAMYAPPISGQITCSSNITGLLLTRDGGKDNNTNDTTEIFRPAERNMKDNWRSELYKYKVVKIEPLGVAPTKAKRRVVQREKRAVGTLRAMFLGFLGAAGSTMGAASMTLTVQARLLLSGIVQQQNNLLKAIEAQQHLLQLTVWGIKQLQARVLAVERYLKDQQLLGIWGCSGKLICTTAVPWNTSWSNRSLDMIWNNMTWMQWEKEVSNYTSLIYTLIEESQNQQEKNEQELLELDKWASLWTWFDITNWLWYIKIFIMVVGGLIGLRIIFAVLSIVNRVRQGYSPLSFQTLLPAPRGPDRPGGIEEEGGERDRGRSDRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 23 - DNA, 2046 bp, Human immunodeficiency virus
atgagagtgaaggagaagtatcagcacttgtggagatgggggtggaaatggggcaccatgctccttgggatattgatgatctgtagtgctacagaaaaattgtgggtcacagtctattatggggtacctgtgtggaaggatgctaacaccactctattttgtgcatcaaatgctaaagcatatgatacagaggcacataatgtttgggccacacacgcctgtgtacccacagaccccaacccacaagaagtagtattgatcaatgtgacagaaaattttaacatgtggaaaaataacatggtagaacaaatgcatgaggatataatcagtttatgggatcaaagcctaaagccatgtgtaaaattaaccccactctgtgttactttaaattgcagtgatgtaaatggcactagtagtaataccactagagagataagaaatgagacaataatggaaaaaggagaagtgaaaaactgctctttcaatatcaccacaagcataagagatagggtgcagaaacaatatgcaacattttatagacttgatgtagtgcagatagatgataatgataatactagttataggatgacaagttgtaacgcctcagtcattacacaggcctgtccaaagatatcctttgagccaattcccatacatttttgtgccccggctggttttgcgattctaaagtgtacagaaaaggggttcaatggaacaggaaaatgtaaaaatgtcagcacagtacaatgtacacatggaattaggccagtagtatcaactcaattgctgttaaatggcagtctagcagaagaaggggtagtaattcgatctcaaaatttaacagacaatactaaaaccataatagtacagctggaaaaagctataaaaattaattgtacaagacccaacaacaatacaagaaaaagtatgagtctaggtccagggaaagtattttatacaacaggacaaataataggagatataagaaaagcacattgtaaccttactgcaggagattggtatagcgctttaaaagagatagccataaaattaagaggacaatttaacaataaaacaatagtctttaagaactcctcaggaggggactcagaaattgtaacccacagttttaattgtggaggggaatttttctactgtgactcaacaaaactgtttaattttacttggaatggtactgaaacaaataacactggaggaagtgataaaatcatactcccatgcagaataaaacaaattataaacatgtggcagaaagtaggacaagcaatgtatgcccctcccatccaaggaaaaatttactgttcatcaaatattacagggctactattaacaagagatggtggcatcaacaatgaaactaacaataccgagaccttcagacctggaggaggaaatatgaaggataattggagaagtgaattatataaatataaagtagtacaaattgaaccattaggagtagcacccaccaaggcaaaaagaagagtggtgcagagagaaaccggtgcagtgggcatgggagctttgttccttgggttcttgggagcagcaggaagcactatgggcgcagcgtcaataacgctgacggtacaggccagacaattattgtctggtatagtgcaacagcagaacaatctgctgagggctgttgaggcgcaacagcatatgttgcaactcacagtctggggcatcaagcagctccaggcgagagtcctggctgtggaaagatacctaagggatcaacagctcctggggctctggggttgctctggaaagctcatttgcaccactactgtgccttggaatactagttggagtaataaatctctgaatgagatttgggataacatgacctggaaggaatgggacagagaaattaacaagtacacaaatgtaatatactccttaattgaagaatcgcagaaccagcaagaaaagaatgaacaagaattattggcattagataagtgggcaagtttgtggaattggtttgacataacaaaatggctgtggtatataaaataa

SEQ ID NO: 24 - protein, 681 aa, Human immunodeficiency virus
MRVKEKYQHLWRWGWKWGTMLLGILMICSATEKLWVTVYYGVPVWKDANTTLFCASNAKAYDTEAHNVWATHACVPTDPNPQEVVLINVTENFNMWKNNMVEQMHEDIISLWDQSLKPCVKLTPLCVTLNCSDVNGTSSNTTREIRNETIMEKGEVKNCSFNITTSIRDRVQKQYATFYRLDVVQIDDNDNTSYRMTSCNASVITQACPKISFEPIPIHFCAPAGFAILKCTEKGFNGTGKCKNVSTVQCTHGIRPVVSTQLLLNGSLAEEGVVIRSQNLTDNTKTIIVQLEKAIKINCTRPNNNTRKSMSLGPGKVFYTTGQIIGDIRKAHCNLTAGDWYSALKEIAIKLRGQFNNKTIVFKNSSGGDSEIVTHSFNCGGEFFYCDSTKLFNFTWNGTETNNTGGSDKIILPCRIKQIINMWQKVGQAMYAPPIQGKIYCSSNITGLLLTRDGGINNETNNTETFRPGGGNMKDNWRSELYKYKVVQIEPLGVAPTKAKRRVVQRETGAVGMGALFLGFLGAAGSTMGAASITLTVQARQLLSGIVQQQNNLLRAVEAQQHMLQLTVWGIKQLQARVLAVERYLRDQQLLGLWGCSGKLICTTTVPWNTSWSNKSLNEIWDNMTWKEWDREINKYTNVIYSLIEESQNQQEKNEQELLALDKWASLWNWFDITKWLWYIK

SEQ ID NO: 25 - DNA, 2058 bp, Human immunodeficiency virus
atgagagtgaaggagaagtatcagcacttgtggagatgggggtggaaatggggcaccatgctccttgggatattgatgatctgtagtgctacagaaaaattgtgggtcacagtctattatggggtacctgtgtggaaagaagcaactaccactctattttgtgcatcagatgctaaagcatatgatacagaggtacataatgtttgggccacacatgcctgtgtacccacagaccccaacccacaagaagtagtattgggaaatgtaacagaatattttaacatgtggaaaaatagtatggtggaacagatgcatgaggatataatcagtctatgggatgaaagcttaaagccatgcgtaaaattaaccccactctgtgtttctttaaattgcactgattggaagaatagtactaatacccataataacagttgtggaagtggttcaaacaactgctctttggaaatggagaaaggagaaataaaaaactgctctttcaatatcacctcacgcatgagagataggacgcaggaaaaatatgcacttttttataaacttgatgtagtacaaatagatggtgataagggtaataccagcagctataggttgatacattgtaatacctcagtcattacacaggcctgtccaaaggtatcctttgagccaattcccatacattattgtaccccggctggttttgcgattctaaagtgtaacgataagaggttcaagggaaaaggacaatgtacaaatgtcagcacagtacagtgtacacatggaattaggccagtggtgtcaactcaattgctgttaaatggcagcctagcagaagaggagataataattaggtctgacaatatcacggacaatgctaaaaccataatagtacagctgaatgaatctgtagcaattacatgtgaaagaccaggcaacaatacaagaagaagtatacctataggaccagggagagtattttatacaggagaaataacaggagatataagaaaagcacattgcaatattactaaaaaaggctgggagagcactttaaaaaagatagctagcaagttaaaagaacaatttaataagacaatagtctttaatcaatcctcaggaggggacccagaaattgtaatgcacagttttaattgtagaggggaatttttctactgtgattcagcacagctgtttaatagtacttggccgtctaatagtactggggaaacaaatgacactcaaggaatcaatatcacactcccatgtagaataaaacaaattataaacatgtggcagggagtaggaaaagcaatgtatgcccctcccatcagaggacgaattacatgtacatcaaatattacagggctgctgttaacaagggatggtggtcataacacaagcaaggagaccttcagacctggaggaggaaatatgagggacaattggagaagtgaactatataaatataaagtagtaaaaattgaaccattaggaatagcacccaccaaggcaaagagaagagtggtgcagagagaaaccggtggagtaacaataggagctatgttccttgggttcttgggagcagcaggaagcactatgggcgcagcgtcaatggcgctgacggcacaggccagacaattattgtctggtatagtgcaacagcagagcaatctgctgagggttattgaggtgcaacagcatatgttgcaactcacagtctggggcatcaagcagctccaggcaagagtcctggctgtggaaagatacctaagggatcaacagctcctagggatttggggttgctctggaaaactcatttgcaccactgctgtaccttggaatgctagttggagcaataaatctcaggaggatatttgggaaaacatgacctggatgcagtgggaaaaagaaattgacaattacacagggttaatctacaccttacttgaaaaatcgcagaaccagcaagaaaagaatgaacaagaattattggaattggataagtgggcaggtttgtggaattggtttgacataacacaatggctgtggtatataaaataa

SEQ ID NO: 26 - protein, 685 aa, Human immunodeficiency virus
MRVKEKYQHLWRWGWKWGTMLLGILMICSATEKLWVTVYYGVPVWKEATTTLFCASDAKAYDTEVHNVWATHACVPTDPNPQEVVLGNVTEYFNMWKNSMVEQMHEDIISLWDESLKPCVKLTPLCVSLNCTDWKNSTNTHNNSCGSGSNNCSLEMEKGEIKNCSFNITSRMRDRTQEKYALFYKLDVVQIDGDKGNTSSYRLIHCNTSVITQACPKVSFEPIPIHYCTPAGFAILKCNDKRFKGKGQCTNVSTVQCTHGIRPVVSTQLLLNGSLAEEEIIIRSDNITDNAKTIIVQLNESVAITCERPGNNTRRSIPIGPGRVFYTGEITGDIRKAHCNITKKGWESTLKKIASKLKEQFNKTIVFNQSSGGDPEIVMHSFNCRGEFFYCDSAQLFNSTWPSNSTGETNDTQGINITLPCRIKQIINMWQGVGKAMYAPPIRGRITCTSNITGLLLTRDGGHNTSKETFRPGGGNMRDNWRSELYKYKVVKIEPLGIAPTKAKRRVVQRETGGVTIGAMFLGFLGAAGSTMGAASMALTAQARQLLSGIVQQQSNLLRVIEVQQHMLQLTVWGIKQLQARVLAVERYLRDQQLLGIWGCSGKLICTTAVPWNASWSNKSQEDIWENMTWMQWEKEIDNYTGLIYTLLEKSQNQQEKNEQELLELDKWAGLWNWFDITQWLWYIK

SEQ ID NO: 27 - DNA, 2058 bp, Human immunodeficiency virus
  misc_feature (1823)..(1823): n is a, c, g, or t
atgagagtgaaggagaagtatcagcacttgtggagatgggggtggaaatggggcaccatgctccttgggatattgatgatctgtagtgctacagaaaaattgtgggtcacagtctattatggggtacctgtgtggaaagaagcaactaccactctattttgtgcatcagatgctaaagcatatgatacagaggtacataatgtttgggccacacatgcctgtgtacccacagaccccaacccacaagaagtagtattgggaaatgtaacagaatattttaacatgtggaaaaatagtatggtggaacagatgcatgaggatataatcagtctatgggatgaaagcttaaagccatgtgtaaaattaaccccactctgtgtttctttaaattgcactgattggaagaatagtactaatacccataataacagttgtggaagtggttcaaacaactgctctttggaaatggagaaaggagaaataaaaaactgctctttcaatatcacctcacgcatgagagataggacgcaggaaaaatatgcacttttttataaacttgatgtagtacaaatagatggtgataagggtaataccagcagctataggttgatacattgtaatacctcagtcattacacaggcctgtccaaaggtatcctttgagccaattcccatacattattgtaccccggctggttttgcgattctaaagtgtaacgataagaggttcaagggaaaaggacaatgtacaaatgtcagcacagtacagtgtacacatggaattaggccagtggtgtcaactcaattgctgttaaatggcagcctagcagaagaggggataataattaggtctgacaatatcacggacaatgctaaaaccataatagtacagctgaatgaatctgtagcaattacatgtgaaagaccaggcaacaatacaagaagaagtatacctataggaccagggagagtattttatacaggagaaataacaggagatataagaaaagcacattgcaatattactaaaaaaggctgggagagcactttaaaaaagatagctagcaagttaaaagaacaatttaataagacaatagtctttaatcaatcctcaggaggggacccagaaattgtaatgcacagttttaattgtagaggggaatttttctactgtgattcagcacagctgtttaatagtacttggccgtctaatagtactggggaaacaaatgacactcaaggaatcaatatcacactcccatgtagaataaaacaaattataaacatgtggcagggagtaggaaaagcaatgtatgcccctcccatcagaggacgaattacatgtacatcaaatattacagggctgctgttaacaagggatggtggtcataacacaagcaaggagaccttcagacctggaggaggaaatatgagggacaattggagaagtgaactatataaatataaagtagtaaaaattgaaccattaggaatagcacccaccaaggcaaagagaagagtggtgcagagagaaaccggtggagtaacaataggagctatgttccttgggttcttgggagcagcaggaagcactatgggcgcagcgtcaatggcgctgacggcacaggccagacaattattgtctggtatagtgcaacagcagagcaatctgctgagggctattgaggcgcaacagcatatgttgcaactcacagtctggggcatcaagcagctccaggcaagagtcctggctgtggaaagatacctaagggatcaacagctcctagggatttggggttgctctggaaaactcatttgcaccantgctgtgccttggaatgctagttggagcaataaatctcaggaggatatttgggaaaacatgacctggatgcagtgggaaaaagaaattgacaattacacagggttaatctacaccttacttgaaaaatcgcagaaccagcaagaaaagaatgaacaagaattattggaattggataagtgggcaggtttgtggaattggtttgacataacacaatggctgtggtatataaaataa

SEQ ID NO: 28 - protein, 685 aa, Human immunodeficiency virus
  misc_feature (608)..(608): Xaa can be any naturally occurring amino acid
MRVKEKYQHLWRWGWKWGTMLLGILMICSATEKLWVTVYYGVPVWKEATTTLFCASDAKAYDTEVHNVWATHACVPTDPNPQEVVLGNVTEYFNMWKNSMVEQMHEDIISLWDESLKPCVKLTPLCVSLNCTDWKNSTNTHNNSCGSGSNNCSLEMEKGEIKNCSFNITSRMRDRTQEKYALFYKLDVVQIDGDKGNTSSYRLIHCNTSVITQACPKVSFEPIPIHYCTPAGFAILKCNDKRFKGKGQCTNVSTVQCTHGIRPVVSTQLLLNGSLAEEGIIIRSDNITDNAKTIIVQLNESVAITCERPGNNTRRSIPIGPGRVFYTGEITGDIRKAHCNITKKGWESTLKKIASKLKEQFNKTIVFNQSSGGDPEIVMHSFNCRGEFFYCDSAQLFNSTWPSNSTGETNDTQGINITLPCRIKQIINMWQGVGKAMYAPPIRGRITCTSNITGLLLTRDGGHNTSKETFRPGGGNMRDNWRSELYKYKVVKIEPLGIAPTKAKRRVVQRETGGVTIGAMFLGFLGAAGSTMGAASMALTAQARQLLSGIVQQQSNLLRAIEAQQHMLQLTVWGIKQLQARVLAVERYLRDQQLLGIWGCSGKLICTXAVPWNASWSNKSQEDIWENMTWMQWEKEIDNYTGLIYTLLEKSQNQQEKNEQELLELDKWAGLWNWFDITQWLWYIK

SEQ ID NO: 29 - DNA, 2046 bp, Human immunodeficiency virus; identical to SEQ ID NO: 23

SEQ ID NO: 30 - protein, 681 aa, Human immunodeficiency virus; identical to SEQ ID NO: 24

SEQ ID NO: 31 - DNA, 2049 bp, Human immunodeficiency virus
atgagagtgaaggagaagtatcagcacttgtggagatgggggtggaaatggggcaccatgctccttgggatattgatgatctgtagtgctacagaaaaattgtgggtcacagtctattatggggtacctgtgtggaaagaagcaaataccactctattttgtgcatcagatgctaaagcatatgattcagaggtacataatgtttgggctacacatgcctgtgtacccacagaccccaacccacaagaagtattattgggaaatgtgacagaaaattttaatatgtggaaaaataacatggtagaacagatgcatgaagatataatcagtttatgggatcaaagcctgaagccatgtgtaaaattaaccccactctgtgttactttaaattgcactgatttgattagcactgagtcggagaatattactgatgccattaatgagaaaggagaaataaaaaactgctcgttcaatatcaccacaagagtaggaaataagttgaagagagaatatgcatttttttataaccttgatgtcgtatcaataaatgatgacaataacaataatacttataggttgataaattgtaatacctcagtcattacacaggcctgtccaaaggtatcctttgagccaattcctatacattattgtgccccggctggttttgcgattctaaagtgtaacgataagaaattcaatggaaaaggacaatgtacaaatgtcagcacagtacaatgtacacatggaattaggccagtagtgtcaactcaactgctgttaaatggcagtctagcagaagaagaagtagtaattagatctgataatatcacgaataatgttaaaatcataatagtacagctgaatgaatctgtagaaatcaagtgtgcaagacccaacaacaatacaagaaaaagtatacctgtaggaccagggcgagcaatttatgcgacaggagacataataggagatataagacaagcacattgtaacattagtagggcacaatggaataacactttaaaacagatagcaagaaaactaagggaacaattcaacaataaaacaatagtgtttaagccatcctcaggaggggacccagaaattgtaatgcacagttttaattgtagaggggaatttttctactgtaattcaacacaactgtttaatagtacttggaatgaaactgctgggaatgatactatagggtcagataacacaactatcacactcccatgcagaataaaacaaattataaacaggtggcaggaagtaggaaaagcaatgtatgcccctcccatcagtggacaaattacatgctcatcaaatattacagggctactattaacaagggatggtggtaaagataataacacaaatgacactactgagatcttcagacctgcagggggaaatatgagggacaattggagaagtgagttatataaatataaagtagtaaaaattgaaccattaggagtagcacccaccaaggcaaagagaagagtggtgcagagagaaaccggtgcagtgggaacattaggagctatgttccttgggttcttgggagcagcaggaagcactatgggcgcagcgtcaatgacgctgacggtacaggccagactattattgtctggtatagtgcaacagcagaacaatttgctgaaagctattgaggcgcaacagcatctgttgcaactcacagtctggggcatcaagcagctccaggcaagagtcctggctgtggaaagatacctaaaggatcaacagctcctagggatttggggttgctctggaaagctcatttgcaccactgctgtgccttggaatactagttggagtaatagatctctggatatgatttggaataacatgacctggatgcagtgggaaaaagaagttagcaattacacaagtttaatatacaccttaattgaagaatcgcaaaaccaacaagaaaagaatgaacaagaattattggaattagataaatgggcaagtttgtggacttggtttgacataacaaactggctgtggtatataaaataa

SEQ ID NO: 32 - protein, 682 aa, Human immunodeficiency virus
MRVKEKYQHLWRWGWKWGTMLLGILMICSATEKLWVTVYYGVPVWKEANTTLFCASDAKAYDSEVHNVWATHACVPTDPNPQEVLLGNVTENFNMWKNNMVEQMHEDIISLWDQSLKPCVKLTPLCVTLNCTDLISTESENITDAINEKGEIKNCSFNITTRVGNKLKREYAFFYNLDVVSINDDNNNNTYRLINCNTSVITQACPKVSFEPIPIHYCAPAGFAILKCNDKKFNGKGQCTNVSTVQCTHGIRPVVSTQLLLNGSLAEEEVVIRSDNITNNVKIIIVQLNESVEIKCARPNNNTRKSIPVGPGRAIYATGDIIGDIRQAHCNISRAQWNNTLKQIARKLREQFNNKTIVFKPSSGGDPEIVMHSFNCRGEFFYCNSTQLFNSTWNETAGNDTIGSDNTTITLPCRIKQIINRWQEVGKAMYAPPISGQITCSSNITGLLLTRDGGKDNNTNDTTEIFRPAGGNMRDNWRSELYKYKVVKIEPLGVAPTKAKRRVVQRETGAVGTLGAMFLGFLGAAGSTMGAASMTLTVQARLLLSGIVQQQNNLLKAIEAQQHLLQLTVWGIKQLQARVLAVERYLKDQQLLGIWGCSGKLICTTAVPWNTSWSNRSLDMIWNNMTWMQWEKEVSNYTSLIYTLIEESQNQQEKNEQELLELDKWASLWTWFDITNWLWYIK

SEQ ID NO: 33 - DNA, 2222 bp, Human immunodeficiency virus
atgagagtgaaggagaagtatcagcacttgtggagatgggggtggaaatggggcaccatgctccttgggatattgatgatctgtagtgctacagaaaaattgtgggtcacagtctattatggggtacctgtgtggaaagaagcaaataccactctattttgtgcatcagatgctaaagcatatgattcagaggtacataatgtttgggctacacatgcctgtgtacccacagaccccaacccacaagaagtattattgggaaatgtgacagaaaattttaatatgtggaaaaataacatggtaaaacagatgcatgaagatataatcagtttatgggatcaaagcctaaagccatgtgtaaaattaaccccactctgtgttactttaaattgcactgatttgattagcactgagtcggagaatattactgatgccattaatgagaaaggagaaataaaaaactgctcgttcaatatcaccacaagagtaggaaataagttgaagagagaatatgcatttttttataaccttgatgtagtatcaataaataatgacaataacaataatacttataggttgataaattgtaatacctcagtcattacacaggcctgtccaaaggtatcctttgagccaattcctatacattattgtgccccggctggttttgcgattctaaagtgtaacgataagaaattcaatggaaaaggacaatgtacaaatgtcagcacagtacaatgtacacatggaattaggccagtagtgtcaactcaactgctgttaaatggcagtctagcagaagaagaagtagtaattagatctgataatatcacgaataatgttaaaatcataatagtacagctgaatgaatctgtagaaatcaagtgtgcaagacccaacaacaatacaagaaaaagtatacctgtaggaccagggcgagcaatttatgcaacaggagacataataggagatataagacaagcacattgtaacattagtagagcacaatggaataacactttaaaacagatatcaagaaaactaagggaacaattcaacaataaaacaatagtgtttaagccatcctcaggaggggacccagaaattgtaatgcacagttttaattgtagaggggaatttttctactgtaattcaacacaactgtttaatagtacttggaatgaaactgctgggaatgatactatagggtcagataacacaactatcacactcccatgcagaataaaacaaattataaacaggtggcaggaagtaggaaaagcaatgtatgcccctcccatcagtggacaaattacatgctcatcaaatattacagggctactattaacaagggatggtggtaaagataataacacaaatgacactactgagatcttcagacctgcagagagaaatatgaaggacaattggagaagtgagttatataaatataaagtagtaaaaattgaaccattaggagtagcacccaccaaggcaaagagaagagtggtgcagagagaaaccggtgcagtgggaacattaagagctatgttccttgggttcttgggagcagcgggaagcactatgggcgcagcgtcaatgacgctgacggtacaggccagactattattgtctggtatagtgcaacagcagaacaatttgctgaaagctattgaggcgcaacagcatctgttgcaactcacagtctggggcatcaagcagctccaggcaagagtcctggctgtggaaagatacctaaaggatcaacagctcctagggatttggggttgctctggaaagctcatttgcaccactgctgtgccctggaatactagttggagtaatagatctctggatatgatttggaataacatgacctggatgcagtgggaaaaagaagttagcaattacacaagtttaatatacaccttaattgaagaatcgcaaaaccaacaagaaaagaatgaacaagaattattagaattagataaatgggcaagtttgtggacttggtttgacataacaaactggctgtggtatataaaataagctagcatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacag

SEQ ID NO: 34 - protein, 682 aa, Human immunodeficiency virus
MRVKEKYQHLWRWGWKWGTMLLGILMICSATEKLWVTVYYGVPVWKEANTTLFCASDAKAYDSEVHNVWATHACVPTDPNPQEVLLGNVTENFNMWKNNMVKQMHEDIISLWDQSLKPCVKLTPLCVTLNCTDLISTESENITDAINEKGEIKNCSFNITTRVGNKLKREYAFFYNLDVVSINNDNNNNTYRLINCNTSVITQACPKVSFEPIPIHYCAPAGFAILKCNDKKFNGKGQCTNVSTVQCTHGIRPVVSTQLLLNGSLAEEEVVIRSDNITNNVKIIIVQLNESVEIKCARPNNNTRKSIPVGPGRAIYATGDIIGDIRQAHCNISRAQWNNTLKQISRKLREQFNNKTIVFKPSSGGDPEIVMHSFNCRGEFFYCNSTQLFNSTWNETAGNDTIGSDNTTITLPCRIKQIINRWQEVGKAMYAPPISGQITCSSNITGLLLTRDGGKDNNTNDTTEIFRPAERNMKDNWRSELYKYKVVKIEPLGVAPTKAKRRVVQRETGAVGTLRAMFLGFLGAAGSTMGAASMTLTVQARLLLSGIVQQQNNLLKAIEAQQHLLQLTVWGIKQLQARVLAVERYLKDQQLLGIWGCSGKLICTTAVPWNTSWSNRSLDMIWNNMTWMQWEKEVSNYTSLIYTLIEESQNQQEKNEQELLELDKWASLWTWFDITNWLWYIK

SEQ ID NO: 35 - DNA, 2571 bp, Human immunodeficiency virus
atgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctataagttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccaacaacaatacaagaaaaagaatccgtatccagagaggaccagggagagcatttgttacaataggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataa

SEQ ID NO: 36 - protein, 872 aa, Human immunodeficiency virus
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILLRHIPRRIRQGLERILL

SEQ ID NO: 37 - protein, 702 aa, Human immunodeficiency virus
MKVKGIRKNYQHLWKWGIMLLGMLMICSAVENLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLRNVTNINNSSEGMRGEIKNCSFNITTSIRDKVKKDYALFYRLDVVPIDNDNTSYRLINCNTSTITQACPKVSFEPIPIHYCTPAGFAILKCKDKKFNGTGPCKNVSTVQCTHGIRPVVSTQLLLNGSLAEEEVVIRSSNFTDNAKNIIVQLKESVEINCTRPNNNTRKSIHIGPGRAFYTTGDIIGDIRQAHCNISRTKWNNTLNQIATKLKEQFGNNKTIVFNQSSGGDPEIVMHSFNCGGEFFYCNSTQLFNSTWNFNGTWNLTQSNGTEGNDTITLPCRIKQIINMWQEVGKAMYAPPIRGQIRCSSNITGLILTRDGGNNHNNDTETFRPGGGDMRDNWRSELYKYKVVKIEPLGVAPTKAKRRVVQRETGAVGTIGAMFLGFLGAAGSTMGAASITLTVQARLLLSGIVQQQNNLLRAIEAQQHLLQLTVWGIKQLQARVLAVERYLRDQQLLGIWGCSGKLICTTAVPWNASWSNKTLDMIWNNMTWMEWEREIDNYTGLIYTLIEESQNQQEKNEQELLELDKWASLWNWFDITNWLWYIKLDKWASLWNWFDITNWLWYIK

SEQ ID NO: 38 - protein, 683 aa, Human immunodeficiency virus
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQRETGAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIK

SEQ ID NO: 39 - protein, 854 aa, Human immunodeficiency virus
MRVKEKYQHLWRWGWKWGTMLLGILMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRDKVQKEYAFFYKLDIVPIDNTSYRLISCNTSVITQACPKVSFEPIPIHYCAPAGFAILKCNNKTFNGTGPCTNVSTVQCTHGIRPVVSTQLLLNGSLAEEDVVIRSANFTDNAKTIIVQLNTSVEINCTRPNNNTRKSIRIQRGPGRAFVTIGKIGNMRQAHCNISRAKWNATLKQIASKLREQFGNNKTIIFKQSSGGDPEIVTHSFNCGGEFFYCNSTQLFNSTWFNSTWSTEGSNNTEGSDTITLPCRIKQFINMWQEVGKAMYAPPISGQIRCSSNITGLLLTRDGGNNNNGSEIFRPGGGDMRDNWRSELYKYKVVKIEPLGVAPTKAKRRVVQREKRAVGIGALFLGFLGAAGSTMGARSMTLTVQARQLLSDIVQQQNNLLRAIEAQQHLLQLTVWGIKQLQARILAVERYLKDQQLLGIWGCSGKLICTTAVPWNASWSNKSLEQIWNNMTWMEWDREINNYTSLIHSLIEESQNQQEKNEQELLELDKWASLWNWFNITNWLWYIKLFIMIVGGLVGLRIVFAVLSIVNRVRQGYSPLSFQTHLPIPRGPDRPEGIEEEGGERDRDRSIRLVNGSLALIWDDLRSLCLFSYHRLRDLLLIVTRIVELLGRRGWEALKYWWNLLQYWSQELKNSAVNLLNATAIAVAEGTDRVIEVLQAAYRAIRHIPRRIRQGLERILL

SEQ ID NO: 40 - protein, 681 aa, Human immunodeficiency virus
MRVKEKYQHLWRWGWKWGTMLLGILMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRDKVQKEYAFFYKLDIVPIDNTSYRLISCNTSVITQACPKVSFEPIPIHYCAPAGFAILKCNNKTFNGTGPCTNVSTVQCTHGIRPVVSTQLLLNGSLAEEDVVIRSANFTDNAKTIIVQLNTSVEINCTRPNNNTRKSIRIQRGPGRAFVTIGKIGNMRQAHCNISRAKWNATLKQIASKLREQFGNNKTIIFKQSSGGDPEIVTHSFNCGGEFFYCNSTQLFNSTWFNSTWSTEGSNNTEGSDTITLPCRIKQFINMWQEVGKAMYAPPISGQIRCSSNITGLLLTRDGGNNNNGSEIFRPGGGDMRDNWRSELYKYKVVKIEPLGVAPTKAKRRVVQRETGAVGIGALFLGFLGAAGSTMGARSMTLTVQARQLLSDIVQQQNNLLRAIEAQQHLLQLTVWGIKQLQARILAVERYLKDQQLLGIWGCSGKLICTTAVPWNASWSNKSLEQIWNNMTWMEWDREINNYTSLIHSLIEESQNQQEKNEQELLELDKWASLWNWFNITNWLWYIK

SEQ ID NO: 41 - protein, 854 aa, Human immunodeficiency virus
MKVKGIRKNYQHLWKWGIMLLGMLMICSAVENLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLRNVTNINNSSEGMRGEIKNCSFNITTSIRDKVKKDYALFYRLDVVPIDNDNTSYRLINCNTSTITQACPKVSFEPIPIHYCTPAGFAILKCKDKKFNGTGPCKNVSTVQCTHGIRPVVSTQLLLNGSLAEEEVVIRSSNFTDNAKNIIVQLKESVEINCTRPNNNTRKSIHIGPGRAFYTTGDIIGDIRQAHCNISRTKWNNTLNQIATKLKEQFGNNKTIVFNQSSGGDPEIVMHSFNCGGEFFYCNSTQLFNSTWNFNGTWNLTQSNGTEGNDTITLPCRIKQIINMWQEVGKAMYAPPIRGQIRCSSNITGLILTRDGGNNHNNDTETFRPGGGDMRDNWRSELYKYKVVKIEPLGVAPTKAKRRVVQREKRAVGTIGAMFLGFLGAAGSTMGAASITLTVQARLLLSGIVQQQNNLLRAIEAQQHLLQLTVWGIKQLQARVLAVERYLRDQQLLGIWGCSGKLICTTAVPWNASWSNKTLDMIWNNMTWMEWEREIDNYTGLIYTLIEESQNQQEKNEQELLELDKWASLWNWFDITNWLWYIKIFIMIVGGLIGLRIVFTVLSIVNRVRQGYSPLSFQTHLPAPRGPDRPEGIEEEGGDRDRDRSVRLVDGFLALFWDDLRSLCLFSYHRLRDLLLIVARIVELLGRRGWEALKYWWNLLQYWSQELRNSAVSLLNATAIAVAEGTDRVIEIVQRIYRAILHIPTRIRQGLERLLL